(19) United States
(12) Patent Application Publication
(10) Pub. No.: US 2009/0069817 A1
(43) Pub. Date: Mar. 12, 2009

(54) INTRASTROMAL CORNEAL MODIFICATION

(75) Inventor: Gholam A. Peyman, New Orleans, LA (US)

Correspondence Address: KNOBBE MARTENS OLSON & BEAR LLP, 2040 MAIN STREET, FOURTEENTH FLOOR, IRVINE, CA, 92614, US

(73) Assignee: AcuFocus, Inc., Irvine, CA (US)

(21) Appl. No.: 12/205,820

(22) Filed: Sep. 5, 2008

Related U.S. Application Data
(63) Continuation of application No. 11/269,926, filed on Nov. 8, 2005.
Application No. 11/269,926 is a continuation-in-part of application No. 09/815,277, filed on Mar. 23, 2001, now Pat. No. 6,989,008.
Application No. 09/815,277 is a continuation-in-part of application No. 09/758,263, filed on Jan. 12, 2001, now abandoned.
Application No. 09/758,263 is a continuation-in-part of application No. 09/397,148, filed on Sep. 16, 1999, now Pat. No. 6,217,571.
Application No. 09/397,148 is a division of application No. 08/569,007, filed on Dec. 7, 1995, now Pat. No. 5,964,748.
Application No. 08/569,007 is a continuation-in-part of application No. 08/552,624, filed on Nov. 3, 1995, now Pat. No. 5,722,971.
Application No. 08/552,624 is a continuation-in-part of application No. 08/546,148, filed on Oct. 20, 1995, now Pat. No. 6,221,067.
Application No. 11/269,926 is a continuation-in-part of application No. 10/784,169, filed on Feb. 24, 2004, now abandoned.
Application No. 10/784,169 is a continuation-in-part of application No. 10/406,558, filed on Apr. 4, 2003, now abandoned.
Application No. 10/406,558 is a continuation-in-part of application No. 10/356,730, filed on Feb. 3, 2003, now abandoned.
Application No. 10/356,730 is a continuation-in-part of application No. 09/843,141, filed on Apr. 27, 2001, now Pat. No. 6,551,307.
(60) Provisional application No. 60/449,617, filed on Feb. 26, 2003.

(51) Int. Cl. A61F 9/008

(52) U.S. Cl. 606/107

(57) ABSTRACT

A method for modifying the curvature of a live cornea to correct a patient's vision. The live cornea is first separated into first and second opposed internal surfaces. Next, a laser beam or a mechanical cutting device can be directed onto one of the first and second internal surfaces, or both, if needed or desired. The laser beam or mechanical cutting device can be then used to incrementally and sequentially ablate or remove a three-dimensional portion of the cornea for making the cornea less curved. An ocular material is then introduced to the cornea to modify the curvature. The ocular material can be either a gel or a solid lens or a combination thereof. In one embodiment, a pocket is formed in the central portion of the cornea to receive an ocular material. In another embodiment, a plurality of internal tunnels are formed in the cornea to receive the ocular material. The ocular material can be either a fluid such as a gel or a solid member. In either case, the ocular material is transparent or translucent, and can have a refractive index substantially the same as the intrastromal tissue of the cornea or a different refractive index from the intrastromal tissue of the cornea.

### CROSS-REFERENCE TO RELATED APPLICATIONS

This application is a continuation of U.S. application Ser. No. 11/269,926, filed Nov. 8, 2005, which is a continuation-in-part of U.S. application Ser. No. 09/815,277, filed Mar. 23, 2001, now U.S. Pat. No. 6,989,008. Said U.S. application Ser. No. 11/269,926, filed Nov. 8, 2005 is also a continuation-in-part of U.S. application Ser. No. 09/758,263, filed Jan. 12, 2001, which a continuation-in-part of U.S. patent application Ser. No. 09/397,148, filed Sep. 16, 1999, now U.S. Pat. No. 6,217,571, which is a divisional application of U.S. patent application Ser. No. 08/569,007, filed Dec. 7, 1995, now U.S. Pat. No. 5,964,748, which is a continuation-in-part of applicant's application Ser. No. 08/552,624, filed Nov. 3, 1995, now U.S. Pat. No. 5,722,971, which is a continuation-in-part of application Ser. No. 08/546,148, filed Oct. 20, 1995, now U.S. Pat. No. 6,221,067. Said U.S. application Ser. No. 11/269,926, filed Nov. 8, 2005 is also a continuation-in-part of U.S. patent application Ser. No. 10/784,169, filed Feb. 24, 2004, which is a continuation-in-part of U.S. patent application Ser. No. 10/406,558, filed Apr. 4, 2003 which claims the benefit of U.S. Provisional Application Ser. No. 60/449,617, filed Feb. 26, 2003, and which is a continuation-in-part of U.S. patent application Ser. No. 10/356,730, filed Feb. 3, 2002 which is a continuation-in-part of U.S. patent application Ser. No. 09/843,141, filed Apr. 27, 2001, now U.S. Pat. No. 6,551,307; the entire contents of each of the above-identified applications is herein incorporated by reference.

### BACKGROUND OF THE INVENTION

1. Field of the Disclosure

The invention relates to a method and apparatus for modifying a live cornea via injecting or implanting optical material in the cornea. In particular, the live cornea is modified by the steps of separating an internal area of the live cornea into first and second opposed radially directed internal surfaces, introducing transparent optical material between the surfaces and then recombining the first and second internal surfaces.

2. Background of the Disclosure

A normal ametropic eye includes a cornea, lens and retina. The cornea and lens of a normal eye cooperatively focus light entering the eye from a far point, i.e., infinity, onto the retina. However, an eye can have a disorder known as ametropia, which is the inability of the lens and cornea to focus the far point correctly on the retina. Typical types of ametropia are myopia, hypertrophic or hyperopia, astigmatism and presbyopia.

A myopic eye has either an axial length that is longer than that of a normal ametropic eye, or a cornea or lens having a refractive power stronger than that of the cornea and lens of an ametropic eye. This stronger refractive power causes the far point to be projected in front of the retina.

Conversely, a hypennetropic or hyperopic eye has an axial lens shorter than that of a normal ametropic eye, or a lens or cornea having a refractive power less than that of a lens and cornea of an ametropic eye. This lesser refractive power causes the far point to be focused on the back of the retina.

An eye suffering from astigmatism has a defect in the lens or shape of the cornea. Therefore, an astigmatic eye is incapable of sharply focusing images on the retina.

In order to compensate for the above deficiencies, optical methods have been developed which involve the placement of lenses in front of the eye (for example, in the form of glasses or contact lenses). However, this technique is often ineffective in correcting severe vision disorders.

An alternative technique is surgery. For example, in a technique known as myopic keratomileucis, a microkeratome is used to cut away a portion of the front of the live cornea from the main section of the live cornea. That cut portion of the cornea is then frozen and placed in a cyrolathe where it is cut and reshaped. Altering the shape of the cut portion of the cornea changes the refractive power of this cut portion, which thus effects the location at which light entering the cut portion of the cornea is focused. The reshaped cut portion of the cornea is then reattached to the main portion of the live cornea. Hence, this reshaped cornea will change the position at which the light entering the eye through the cut portion is focused, so that the light is focused more precisely on the retina, thus remedying the ametropic condition.

Keratophakia is another known surgical technique for correcting severe ametropic conditions of the eye by altering the shape of the eye's cornea. In this technique, an artificial organic or synthetic lens is implanted inside the cornea to thereby alter the shape of the cornea and thus change its refractive power. Accordingly, as with the myopic keratomileucis technique, it is desirable that the shape of the cornea be altered to a degree which enables light entering the eye to be focused correctly on the retina.

A further known surgical technique is radial keratotomy. This technique involves cutting numerous slits in the front surface of the cornea to alter the shape of the cornea and thus, alter the refractive power of the cornea. It is desirable that the altered shape of the cornea enables light entering the eye to be focused correctly on the retina. However, this technique generally causes severe damage to the Bowman's layer of the cornea, which results in scarring. This damage and scarring results in glare that is experienced by the patient, and also creates a general instability of the cornea. Accordingly, this technique has generally been abandoned by most practitioners.

Laser in situ keratomileusis (LASIK), as described, for example, in U.S. Pat. No. 4,840,175 to Peyman, the entire contents of which is incorporated herein by reference, is a further known surgical technique for correcting severe ametropic conditions of the eye by altering the shape of the eye's cornea. In the LASIK technique, a motorized blade is used to separate a thin layer of the front of the cornea from the remainder of the cornea in the form of a flap. The flap portion of the cornea is lifted to expose an inner surface of the cornea. The exposed inner surface of the cornea is irradiated with laser light, ablated and thus reshaped by the laser light. The flap portion of the cornea is then repositioned over the reshaped portion and allowed to heal.

In the LASIK technique, it is critical that the tissue ablation is made with an excimer laser, which is difficult to operate and is very expensive. In addition, the process requires tissue removal which might lead to thinning of the cornea or ectasia, which is abnormal bulging of the cornea that can adversely affect vision.

In all of the above techniques, it is necessary that the cornea be prevented from moving while the cutting or separating of the corneal layers is being performed. Also, it is necessary to flatten out the front portion of the cornea when the corneal layers are being separated or cut so that the separation or cut between the layers can be made at a uniform distance from the front surface of the cornea. Previous techniques for flatting out the front surface of the cornea involve applying pressure to the front surface of the cornea with an instrument such as a flat plate.

In addition to stabilizing the cornea when the cutting or separating is being performed, the cutting tool must be accurately guided to the exact area at which the cornea is to be cut. Also, the cutting tool must be capable of separating layers of the cornea without damaging those layers or the surrounding layers.

Furthermore, when the keratotomy technique is being performed, it is desirable to separate the front layer from the live cornea so that the front layer becomes a flap-like layer that is pivotally attached to the remainder of the cornea and which can be pivoted to expose an interior layer of the live cornea on which the implant can be positioned or which can be ablated by the laser. However, these methods disturb the optical axis of the eye, which passes through the center of the front-portion of the cornea and extends longitudinally through the eye. Care also must be taken so as not to damage the Bowman's layer of the eye.

In addition, because the epithelial cells which are present on the surface of the live cornea may become attached to the blade when the blade is being inserted into the live cornea and thus become lodged between the layers of the live cornea, thereby clouding the vision of the eye, it is desirable to remove the epithelium cells prior to performing the cutting.

Examples of known apparatuses for cutting incisions in the cornea and modifying the shape of the cornea are described in U.S. Pat. No. 5,964,776 to Peyman, U.S. Pat. No. 5,919,185 to Peyman, U.S. Pat. No. 5,722,971 to Peyman, U.S. Pat. No. 4,298,004 to Schachar et al., U.S. Pat. No. 5,215,104 to Steinert, and U.S. Pat. No. 4,903,695 to Warner.

In an ametropic human eye, the far point, i.e., infinity, is focused on the retina. Ametropia results when the far point is projected either in front of the retina, i.e., myopia, or in the back of this structure, i.e., hypermetropic or hyperopic state.

In a myopic eye, either the axial length of the eye is longer than in a normal eye, or the refractive power of the cornea and the lens is stronger than in ametropic eyes. In contrast, in hypermetropic eyes the axial length may be shorter than normal or the refractive power of the cornea and lens is less than in a normal eye. Myopia begins generally at the age of 5-10 and progresses up to the age of 20-25. High myopia greater than 6 diopter is seen in 1-2% of the general population. The incidence of low myopia of 1-3 diopter can be up to 10% of the population.

The incidence of hypermetropic eye is not known. Generally, all eyes are hypermetropic at birth and then gradually the refractive power of the eye increases to normal levels by the age of 15. However, a hypermetropic condition is produced when the crystalline natural lens is removed because of a cataract.

Correction of myopia is achieved by placing a minus or concave lens in front of the eye, in the form of glasses or contact lenses to decrease the refractive power of the eye. The hypermetropic eye can be corrected with a plus or convex set of glasses or contact lenses. When hypermetropia is produced because of cataract extraction, i.e., removal of the natural crystalline lens, one can place a plastic lens implant in the eye, known as an intraocular lens implantation, to replace the removed natural crystalline lens.

Surgical attempts to correct myopic ametropia dates back to 1953 when Sato tried to flatten the corneal curvature by performing radial cuts in the periphery of a corneal stroma (Sato, Am. J. Opthalmol. 36:823, 1953). Later, Fyoderov (Ann. Opthalmol. 11:1185, 1979) modified the procedure to prevent postoperative complications due to such radial keratotomy. This procedure is now accepted for correction of low myopia for up to 4 diopter (See Schachar [eds] Radial Keratotomy LAL, Pub. Denison, Tex., 1980 and Sanders D [ed] Radial Keratotomy, Thorofare, N.J., Slack publication, 1984).

Another method of correcting myopic ametropia is by lathe cutting of a frozen lamellar corneal graft, known as myopic keratomileusis. This technique may be employed when myopia is greater than 6 diopter and not greater than 18 diopter. The technique involves cutting a partial thickness of the cornea, about 0.26-0.32 mm, with a microkeratome (Barraquer, Opthalmology Rochester 88:701, 1981). This cut portion of the cornea is then placed in a cryolathe and its surface modified. This is achieved by cutting into the corneal parenchyma using a computerized system. Prior to the cutting, the corneal specimen is frozen to −18° F. The difficulty in this procedure exists in regard to the exact centering of the head and tool bit to accomplish the lathing cut. It must be repeatedly checked and the temperature of the head and tool bit must be exactly the same during lathing. For this purpose, carbon dioxide gas plus fluid is used. However, the adiabatic release of gas over the carbon dioxide liquid may liberate solid carbon dioxide particles, causing blockage of the nozzle and inadequate cooling.

The curvature of the corneal lamella and its increment due to freezing must also be calculated using a computer and a calculator. If the corneal lamella is too thin, this results in a small optical zone and a subsequent unsatisfactory correction. If the tissue is thicker than the tool bit, it will not meet at the calculated surface resulting in an overcorrection.

In addition, a meticulous thawing technique has to be adhered to. The complications of thawing will influence postoperative corneal lenses. These include dense or opaque interfaces between the corneal lamella and the host. The stroma of the resected cornea may also become opaque (Binder Arch Opthalmol 100:101, 1982 and Jacobiec, Opthalmology [Rochester] 88:1251, 1981; and Krumeich J H, Arch, AOO, 1981). There are also wide variations in postoperative uncorrected visual acuity. Because of these difficulties, not many cases of myopic keratomileusis are performed in the United States.

Surgical correction of hypermetropic keratomycosis involves the lamellar cornea as described for myopic keratomileusis. The surface of the cornea is lathe cut after freezing to achieve higher refractive power. This procedure is also infrequently performed in the United States because of the technical difficulties and has the greatest potential for lathing errors. Many ophthalmologists prefer instead an alternative technique to this procedure, that is keratophakia, i.e., implantation of a lens inside the cornea, if an intraocular lens cannot be implanted in these eyes.

Keratophakia requires implantation of an artificial lens, either organic or synthetic, inside the cornea. The synthetic lenses are not tolerated well in this position because they interfere with the nutrition of the overlying cornea. The organic lenticulas, though better tolerated, require frozen lathe cutting of the corneal lenticule.

Problems with microkeratomies used for cutting lamellar cornea are irregular keratectomy or perforation of the eye. The recovery of vision is also rather prolonged. Thus, significant time is needed for the implanted corneal lenticule to clear up and the best corrective visions are thereby decreased because of the presence of two interfaces.

Application of ultraviolet and shorter wavelength lasers also have been used to modify the cornea. These lasers are commonly known as excimer lasers which are powerful sources of pulsed ultraviolet radiation. The active medium of these lasers are composed of the noble gases such as argon, krypton and xenon, as well as the halogen gases such as fluorine and chlorine. Under electrical discharge, these gases react to build excimer. The stimulated emission of the excimer produces photons in the ultraviolet region.

Previous work with this type of laser has demonstrated that far ultraviolet light of argon-fluoride laser light with the wavelength of 193 nm. can decompose organic molecules by breaking up their bonds. Because of this photoablative effect, the tissue and organic and plastic material can be cut without production of heat, which would coagulate the tissue. The early work in opthalmology with the use of this type of laser is reported for performing radial cuts in the cornea in vitro (Trokel, Am. J. Opthalmol 1983 and Cotliar, Opthalmology 1985). Presently, all attempts to correct corneal curvature via lasers are being made to create radial cuts in the cornea for performance of radial keratotomy and correction of low myopia.

Because of the problems related to the prior art methods, there is a continuing need for improved methods to correct eyesight.

### SUMMARY OF THE INVENTION

A device for forming a sub-epithelial flap is presented. The device includes a separating device adapted to separate an epithelial layer of the cornea from a remainder of the cornea, and a rotating device coupled to the separating device and adapted to rotate the separating device in an arcuate path such that the separating device separates the epithelial layer to form a flap that remains attached to the cornea at an area through which the main optical axis passes.

A method of forming a sub-epithelial flap in the cornea of an eye is presented. The method includes the steps of positioning a separating device adjacent the exterior surface of the cornea, and rotating the separating device in an arcuate path such that the separating device separates an epithelial layer from the remainder of the cornea to form a flap that remains attached to the cornea at an area through which the main optical axis passes.

A device for forming a sub-epithelial flap is present. The device includes a separating device adapted to separate an epithelial layer of the cornea from a remainder of the cornea and a rotating means coupled to the separating device and adapted to rotate the separating device in an arcuate path such that the separating device separates the epithelial layer to form a flap that remains attached to the cornea at an area through which the main optical axis passes.

A device for forming a flap in the surface of the cornea of an eye is also present. The device includes a cornea stabilizing device a cutting tool adapted to separate a layer of the corneal epithelial from the remainder of the cornea, exposing at least a portion of the Bowman's layer, and a rotating device coupled to the cutting tool and adapted to rotate the cutting tool in an arcuate path, thereby forming an arcuate flap having an outer edge free of the remainder of the cornea and an inner portion attached to the remainder of the cornea at substantially at an area through which the main optical axis passes.

Additional features and advantages are described herein, and will be apparent from, the following Detailed Description and the figures.

Accordingly, it is a primary object of the present invention to provide a method for modifying corneal curvature via introducing a transparent optical material into an internal portion of the cornea.

Another object of the invention is to provide such a method that can modify the curvature of a live cornea, thereby eliminating the need and complications of working on a frozen cornea.

Another object of the invention is to provide a method for improving eyesight without the use of glasses or contact lenses, but rather by merely modifying the corneal curvature.

Another object of the invention is to provide a method for modifying corneal curvature by using a source of laser light in a precise manner and introducing a transparent optical material into the stroma of the cornea.

Another object of the invention is to provide a method that can modify the curvature of a live cornea without the need of sutures.

Another object of the invention is to provide a method that can modify the curvature of a live cornea with minimal incisions into the epithelium and Bowman's layer of the cornea.

Another object of the invention is to provide a method for modifying the corneal curvature by ablating or coagulating the corneal stroma and introducing a transparent optical material into the stroma of the cornea.

The foregoing objects are basically attained by a method of modifying the curvature of a patient's live cornea comprising the steps of separating an internal area of the live cornea into first and second opposed internal surfaces, the first internal surface facing in the posterior direction and the second internal surface facing in the anterior direction, introducing a transparent optical material between the surfaces, and recombining the first and second internal surfaces, the separating, directing and recombining steps taking place without freezing the cornea. other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring now to the drawings which form a part of this original disclosure:

FIG. 1 is a side view of an apparatus for creating a flap in a live cornea of an eye according to an embodiment of the present invention;

FIG. 2 is an exploded perspective view of the apparatus shown in FIG. 1;

FIG. 3 is a bottom view of the apparatus shown in FIG. 1;

FIG. 4 is a bottom view of the apparatus shown in FIG. 1 with the cornea stabilizing device removed;

FIG. 5 is a cross-sectional view of the apparatus and eye as shown in FIG. 1;

FIG. 6 is a cross-sectional view of an eye having an astigmatic portion;

FIG. 7 is a top view of the eye shown in FIG. 6 into which an incision is being formed by the apparatus shown in FIGS. 1-5;

FIG. 8 is a cross-sectional view of the eye shown in FIG. 6 having a flap formed by the apparatus shown in FIGS. 1-5;

FIG. 9 is a cross-sectional view of the eye shown in FIGS. 6 and 9 with the flap replaced;

FIG. 10 is a cross-sectional view of the eye shown in FIG. 6 with a flap formed as shown in FIG. 9 and additional incisions under the flap;

FIG. 11 is a cross-sectional view of the eye shown in FIG. 6 having a flap fanned therein as shown in FIG. 9, which has been lifted up;

FIG. 12 is a top view of the eye as shown in FIG. 10, with additional incisions made in the exposed surface under the flap;

FIG. 13 is a top view of the eye as shown in FIG. 10, with additional incisions made in the exposed surface under the flap;

FIG. 14 is a top view of the eye as shown in FIG. 10, with tissue shrinkage produced in the exposed surface under the flap;

FIG. 15 is a top view of the eye as shown in FIG. 10, with the combination of incisions and tissue shrinkage made in the exposed surface under the flap;

FIG. 16 is a top view of the eye as shown in FIG. 10, with the combination of incisions and tissue shrinkage made in the exposed surface under the flap;

FIG. 17 is a cross-sectional view of the eye as shown in FIG. 6, with incisions made in the cornea prior to creation of the flap;

FIG. 18 is a cross-sectional view of the eye as shown in FIG. 6, with incisions made in the cornea after to creation of the flap;

FIG. 19 is a side view of an eye used with a flap creating apparatus according to another embodiment of the present invention;

FIG. 20 is a top view of the eye and flap creating apparatus shown in FIG. 19;

FIG. 21 is a schematic illustration of a laser water jet used as the flap creating apparatus as shown in FIGS. 19 and 20 according to an embodiment of the present invention;

FIG. 22 is a top view of the eye shown in FIG. 6 with a cutting device that can be used with the apparatus of FIGS. 1-5 according to another embodiment of the present invention, wherein the device is adapted to form an epithelial flap;

FIG. 23 is a cross sectional view of the cutting device and eye of FIG. 23 illustrating the cutting device separating a layer of epithelium from the cornea; and

FIG. 24 shows an inlay being positioned under the separated layer of epithelium of FIG. 23.

FIG. 25 is a side elevational view in section taken through the center of an eye showing the cornea, pupil and lens;

FIG. 26 is a side elevational view in section similar to that shown in FIG. 25 except that a thin layer has been removed from the front of the cornea, thereby separating the cornea into first and second opposed internal surfaces;

FIG. 27 is a diagrammatic side elevational view of the eye shown in FIG. 26 with a laser beam source, diaphragm and guiding mechanism being located adjacent thereto;

FIG. 28 is a side elevational view in section of an eye that has been treated by the apparatus shown in FIG. 27 with ablation conducted in an annular area spaced from the center of the internal surface on the cornea;

FIG. 29 is a front elevational view of the ablated cornea shown in FIG. 28;

FIG. 30 is a side elevational view in section showing the ablated cornea of FIGS. 28 and 29 with the thin layer previously removed from the cornea replaced onto the ablated area in the cornea, thereby increasing the curvature of the overall cornea;

FIG. 31 is a side elevational view in section of an eye which has been ablated in the central area of the internal surface on the cornea;

FIG. 32 is a front elevational view of the cornea having the central ablated portion shown in FIG. 31;

FIG. 33 is a side elevational view in section of the ablated cornea of FIGS. 31 and 32 in which the thin layer previously removed from the cornea is replaced over the ablated area, thereby reducing the curvature of the overall cornea;

FIG. 34 is a front elevational view of the adjustable diaphragm shown in FIG. 27 used for directing the laser beam towards the eye;

FIG. 35 is a front elevational view of the guiding mechanism shown in FIG. 27 having a rotatable orifice of variable size formed therein, for directing the laser beam towards the eye in a predetermined pattern;

FIG. 36 is a right side elevational view of the guiding mechanism shown in FIG. 35;

FIG. 37 is a right side elevational view in section taken along line 37-37 in FIG. 35 showing the internal parts of the guiding mechanism;

FIG. 38 is a front elevational view of a modified guiding mechanism including a movable orifice;

FIG. 39 is a diagrammatic side elevational view of a second modified guiding mechanism for a laser beam including a universally supported mirror and actuating motors used for moving the mirror and thereby guiding the laser beam in the predetermined pattern;

FIG. 40 is a diagrammatic side elevational view of a third modified guiding mechanism comprising a housing and a rotatable fiber optic cable;

FIG. 41 is an end elevational view of the housing and fiber optic cable shown in FIG. 40;

FIG. 42 is a diagrammatic side elevational view of a laser source, diaphragm and guiding mechanism for use in ablating the thin layer removed from the cornea, which is shown supported by a pair of cups;

FIG. 43 is a front elevational view of a live cornea which has been cut with a spatula to separate the central portion of the cornea into first and second opposed internal surfaces in accordance with the present invention;

FIG. 44 is a side elevational view in section taken along line 44-44 of the cornea shown in FIG. 43;

FIG. 45 is a front elevational view of a cornea that has been cut as shown in FIG. 43 with ablation conducted in the central portion of the cornea by a laser;

FIG. 46 is a side elevational view in section taken along line 46-46 of the cornea shown in FIG. 45;

FIG. 47 is a side elevational view in section taken through the center of an eye showing the ablated cornea of FIGS. 43-46 with the fiber optic tip removed;

FIG. 48 is a side elevational view in section taken through the center of an eye showing the ablated cornea of FIGS. 43-47 in its collapsed position, thereby decreasing the curvature of the central portion of the cornea;

FIG. 49 is an enlarged, partial cross-sectional view of a cornea with a fiber optic tip cutting, separating and ablating the cornea into first and second opposed internal surfaces;

FIG. 50 is an enlarged, partial cross-sectional view of a cornea with a fiber optic tip having an angled end for ablating the cornea;

FIG. 51 is an enlarged, partial cross-sectional view of a cornea with a fiber optic tip having a bent end for ablating the cornea;

FIG. 52 is a front elevational view of a live cornea in which a plurality of radially extending cuts have been made with a spatula to separate the cornea at each of the radially extending cuts into first and second opposed internal surfaces in accordance with the present invention;

FIG. 53 is a front elevational view of a cornea in which the radially extending cuts shown in FIG. 52 have been ablated to create a plurality of radially extending tunnels;

FIG. 54 is a side elevational view in section taken along line 54-54 of the cornea of FIG. 53 with the fiber optic tip removed;

FIG. 55 is a side elevational view in section taken along the center of an eye showing the ablated cornea of FIGS. 52-54 in its collapsed position, thereby decreasing the curvature of the central portion of the cornea;

FIG. 56 is a front elevational view of a live cornea in which a plurality of radially extending cuts have been made with a spatula to separate the cornea at each of the radially extending cuts into first and second opposed internal surfaces in accordance with the present invention;

FIG. 57 is a side elevational view in section taken along line 57-57 of the cornea of FIG. 56 with the spatula removed;

FIG. 59 is a front elevational view of a cornea that has been radially cut as shown in FIGS. 56 and 57 with coagulation conducted at the ends of the radial cuts by a laser, thereby increasing the curvature of the central portion of the cornea;

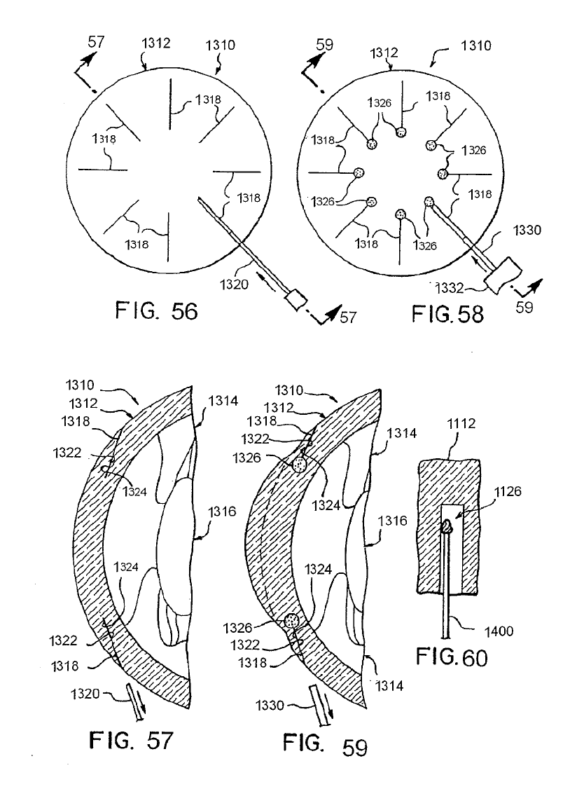

FIG. 56 is a side elevational view in section taken along line 59-59 of the cornea of FIG. 58 with the laser removed and coagulation conducted at the ends of the radial cuts to increase the curvature of the central portion of the cornea;

FIG. 60 is an enlarged, partial cross-sectional view of a cornea with a drill tip removing tissue therefrom;

FIG. 61 is a front elevational view of a live cornea that has been cut to form an intrastromal pocket and showing a tool for injecting or implanting ocular material into the pocket;

FIG. 62 is an enlarged side elevational view in section taken through the center of an eye showing the intrastromal pocket over filled with ocular material thereby increasing the curvature of the central portion of the cornea;

FIG. 63 is an enlarged side elevational view in section taken through the center of an eye showing the intrastromal pocket partially filled with ocular material thereby decreasing the curvature of the central portion of the cornea;

FIG. 64 is an enlarged side elevational view in section taken through the center of an eye showing the intrastromal pocket completely filled with ocular material restoring the curvature of the central portion of the cornea to its original curvature;

FIG. 65 is a rear elevational view of an ocular implant or material in accordance with the present invention for implanting into a cornea;

FIG. 66 is a cross-sectional view of the ocular implant or material illustrated in FIG. 65 taken along section line 66-66;

FIG. 67 is an enlarged side elevational view in section taken through the center of an eye showing the intrastromal pocket with the ocular implant or material of FIGS. 65 and 66 therein for increasing the curvature of the central portion of the cornea;

FIG. 68 is an enlarged side elevational view in section taken through the center of an eye showing the intrastromal pocket with the ocular implant or material of FIGS. 65 and 66 therein for decreasing the curvature of the central portion of the cornea;

FIG. 69 is an enlarged side elevational view in section taken through the center of an eye showing the intrastromal pocket with the ocular implant or material of FIGS. 65 and 66 therein for maintaining the original curvature of the central portion of the cornea;

FIG. 70 is a front elevational view of a live cornea which has been cut to form a plurality of radial tunnels or pockets and showing a tool for injecting or implanting ocular material into the tunnels;

FIG. 71 is an enlarged side elevational view in section taken through the center of the eye showing the radial tunnels or pockets of FIG. 70 overfilled with ocular material thereby modifying the cornea and increasing its curvature;

FIG. 72 is an enlarged side elevational view in section taken through the center of the eye showing the radial tunnels or pockets of FIG. 70 underfilled with ocular material thereby modifying the cornea and decreasing its curvature;

FIG. 73 is an enlarged side elevational view in section taken through the center of the eye showing the radial tunnels or pockets of FIG. 70 completely filled with ocular material thereby modifying the cornea;

FIG. 74 is an enlarged side elevational view in section taken through the center of the eye showing one of the tunnels or pockets overfilled with ocular material to increase the curvature of a selected portion of the cornea and another tunnel or pocket underfilled to decrease the curvature of a selected portion of the cornea;

FIG. 75 is an enlarged side elevational view in section taken through the center of the eye showing one of the tunnels or pockets completely filled with ocular material to maintain a portion of the cornea at its original shape and another tunnel or pocket overfilled with ocular material to increase the curvature of a selected portion of the cornea;

FIG. 76 is an enlarged side elevational view in section taken through the center of the eye showing one of the tunnels or pockets completely filled with ocular material to maintain a portion of the cornea at its original shape and another tunnel or pocket unfilled to collapse or decrease the curvature of a selected portion of the cornea;

FIG. 77 is an enlarged side elevational view in section taken through the center of the eye showing one of the tunnels or pockets overfilled with ocular material to increase the curvature of a selected portion of the cornea and another tunnel or pocket unfilled to collapse or decrease the curvature of a selected portion of the cornea;

FIG. 78 is an exploded side elevational view in section taken through the center of an eye showing a thin layer or portion of the cornea completely removed from the live cornea and the ocular material or implant of FIGS. 65 and 66 positioned between the thin layer and the remainder of the live cornea;

FIG. 79 is an enlarged side elevational view in section taken through the center of the eye showing the ocular implant illustrated in FIGS. 65 and 66 implanted in the cornea with the thin layer of the cornea replaced over the ocular implant to increase the curvature of the cornea;

FIG. 80 is an enlarged side elevational view in section taken through the center of the eye showing the ocular implant illustrated in FIGS. 65 and 66 implanted in the cornea with the thin layer of the cornea replaced over the ocular implant to decrease the curvature of the cornea;

FIG. 81 is an enlarged side elevational view in section taken through the center of the eye showing the ocular implant illustrated in FIGS. 65 and 66 implanted in the cornea with the thin layer of the cornea replaced over the ocular implant to maintain the cornea's original curvature;

FIG. 82 is an enlarged side elevational view in cross section through the center of an eye showing a circular cut or groove in the cornea and the ocular implant of FIGS. 65 and 66 positioned between the separated internal layers, but before the separated internal layers are replaced or rejoined on the cornea;

FIG. 83 is a side elevational view in section through the center of the eye showing the outer surface of the cornea cut to form a flap having a portion still attached to the cornea to expose the intrastromal layers of the cornea;

FIG. 84 is a front elevational view of an ocular implant or material in accordance with the present invention for implanting within the intrastromal area of the cornea; and

FIG. 85 is a cross-sectional view of the ocular implant or material illustrated in FIG. 84 taken along section line 85-85.

### DETAILED DESCRIPTION OF THE PREFERRED EMBODIMENT

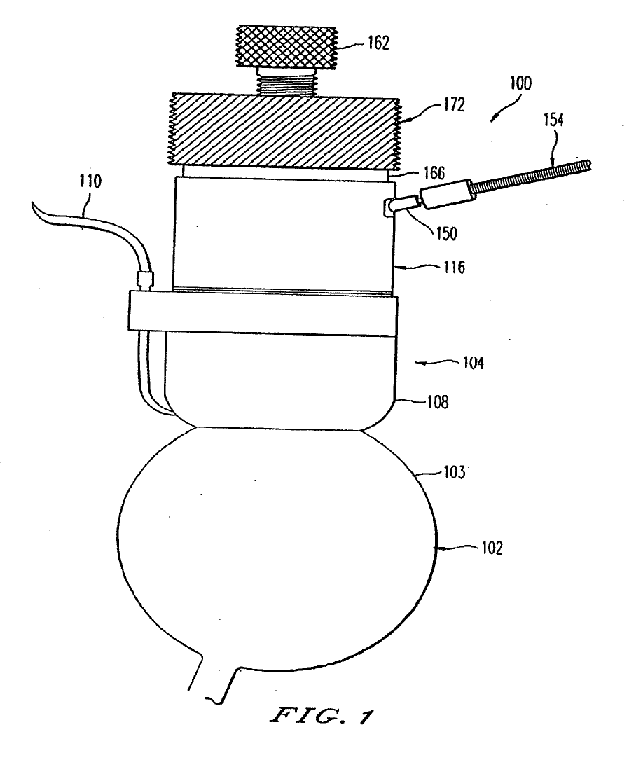

An embodiment of an apparatus 100 for creating a substantially circular flap about the circumference of a live cornea of an eye 102 is illustrated in FIGS. 1-5. Specifically, the apparatus 100 includes a cornea holding apparatus 104 and a cutting mechanism 106.

The cornea holding apparatus 104 includes a cornea receiving section 108 which receives a front portion of a live cornea 103 of a patient's eye 102 as shown, for example, in FIG. 1. Specifically, a tube 110 having an opening 112 therein extending along the length thereof is coupled to the cornea receiving section 108 such that the opening 112 communicates with an interior cavity 114 of the cornea receiving section 108. The interior surface of the cornea receiving section 108 can include a plurality of steps or ridges (not shown) which contact the surface of the live cornea 163 and assist in stabilizing the cornea from movement when the cornea is received in the cornea receiving section 108. That is, as the front surface of the cornea 103 of the eye 102 is received in the receiving section 108, suction will be applied via tube 110 to the internal cavity 114 of the receiving section 108 to suck the cornea into the cavity 114.

As further illustrated, the cutting mechanism 106 includes a cylindrical housing 116 having threads 118 that engage with threads 120 in the inner surface of the cornea holding apparatus 104 to secure the cutting mechanism 106 to the cornea holding apparatus 104. The cylindrical housing 116 includes an opening 122 therein which receives a large cylindrical member 124 having a flange portion 126 that rests on a step 128 in the interior of the cylindrical housing 116.

The large cylindrical member 124 has an opening 130 passing therethrough, into which is received a small cylindrical member 132. The small cylindrical member 132 has a flange portion 134 that rests on a step 136 in the interior of the large cylindrical member 124. Accordingly, the small cylindrical member 132 becomes nested within the large cylindrical member 124. Also, the large and small cylindrical members 124 and 132 remain rotatable with respect to each other and with respect to the cylindrical housing 116.

As further shown, the large cylindrical member 124 includes teeth 138 about its upper circumference, and the small cylindrical member 132 includes teeth 140 about its upper circumference. A gear member 142 includes a gear portion 144 that engages with the teeth 138 and 140 of the large and small cylindrical members 124 and 132, respectively. Gear member 142 further includes a shaft portion 146 that passes through an opening 148 in the cylindrical housing 116 and further through an opening in a support 150 that is screwed to the cylindrical housing 116 by screws 152.

The shaft portion 146 is further received into an opening in a drive shaft 154 which can be manually or mechanically rotated to rotate the gear member 142 as described in more detail below. The shaft portion 146 is secured to the drive shaft 154 by a screw 156 that passes through a hole 158 in the drive shaft 154 and engages with the shaft portion 146 to secure the shaft portion 146 to the drive shaft 154. A blade 160 made of an appropriate material such as surgical steel and having a diamond cutting edge, for example, is coupled to the bottoms of large cylindrical member 124 and small cylindrical member 132 by clips 159 and 161, and is thus rotated when the large and small cylindrical members 124 and 132 are rotated by the gear member 142 as described in more detail below.

The cutting mechanism 106 further includes a clear or substantially clear viewer 162, a viewer mounting portion 164, and a spacer 166. The viewer 162 is preferably a synthetic material, such as an acrylic, plexy glass, or the like, having threads which are as fine as possible. The viewer 162 includes a threaded portion 168 and a shaft portion 170. The shaft portion 170 passes through a threaded opening 172 in the viewer mounting portion 164 so that the threaded portion 168 engaged with the threads in the threaded opening 172. The shaft portion 170 further passes through the opening 133 of small cylindrical member 132, such that the bottom of shaft portion 170 extends toward the bottom of small cylindrical member 132.

The viewer mounting portion 164 further includes threads 174 that engage with threads 176 in the cylindrical housing 116 to secure the viewer mounting portion 164 with the housing 116. Spacer 166 limits the depth to which the viewer mounting portion 164 is received in housing 116. Furthermore, the threaded engagement between threaded opening 172 and threaded portion 168 of the viewer 162 enable the bottom of the shaft portion 170 of the viewer to be raised or lowered as desired by rotating the viewer 162 clockwise or counterclockwise.

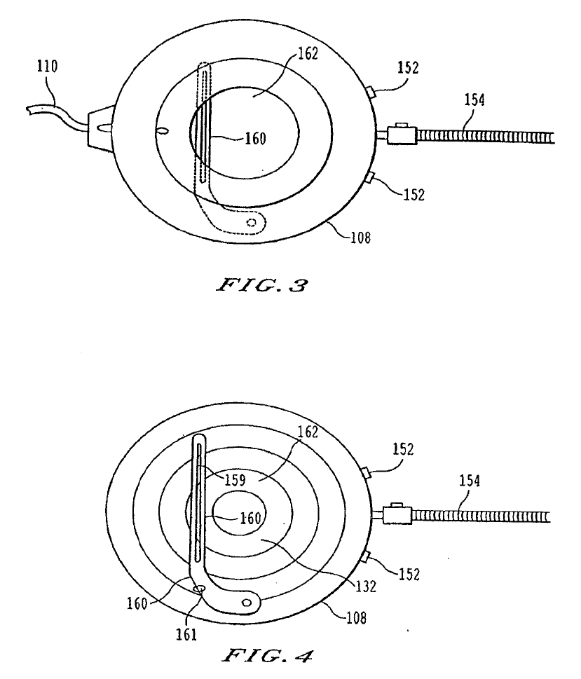

A manner in which the apparatus 100 discussed above is used to correct vision disorders in the eye 102 will now be described. FIG. 6 is a cross section of an eye 102 suffering from astigmatism. Specifically, the front surface of the cornea 103 of the eye 102 has an astigmatic portion 178. The astigmatic portion 178 is a portion of the cornea 103 that is bulged or otherwise misshaped with respect to the remaining front surface of the cornea 103. The apparatus 100 can be used to cut a flap into at least the astigmatic portion 178 of the cornea 103 to correct the astigmatic condition. [0055] The thickness of the cornea 103 is first measured. Then, the front of the eye 102 is placed in the receiving section 108 of the cornea holding apparatus 104 as shown, for example, in FIGS. 1 and 5. A vacuum is applied to tube 110 to create a suction in the cornea receiving section 108 which draws the front of the cornea of the eye 102 toward the bottom of the viewer 162 so that the bottom surface of the viewer 162 flattens the front surface of the cornea as shown, in particular, in FIG. 5. The position of the bottom of the viewer 162 can be adjusted in a manner described above so that when the front portion of the cornea contacts the bottom of the viewer 162, the astigmatic portion 178 of the cornea 103 is aligned with the blade 160.

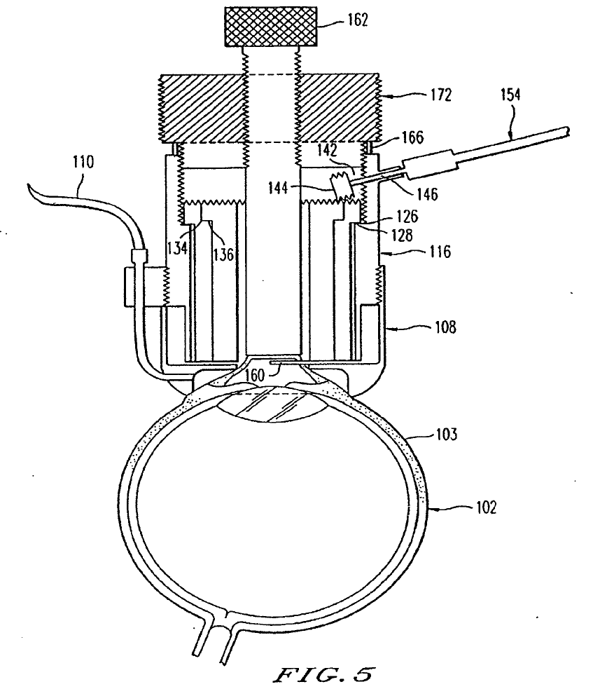

The drive shaft 154 of the cutting mechanism 106 can then be rotated to cut an incision in the cornea of the eye 102 to correct the vision disorder of the eye. For example, as shown in FIGS. 7 and 8, when the drive shaft 154 is rotated, the gear member 142 engages the teeth 138 and 142 of the large and small cylindrical members 124 and 132, respectively, and rotates the large and small cylindrical members 124 and 132. When the large and small cylindrical members 124 and 132 rotate, they move the blade 160 about the circumference of the cornea 103 in a direction along arrow R in FIG. 7 to create an incision in the cornea 103. The drive shaft 154 can be rotated to cause the blade 160 to create an incision only in the astigmatic portion 178 of the cornea 103, or about the entire circumference of a cornea 103 or any portion of the circumference of the cornea 103.

Assuming, for example, that the drive shaft 154 is rotated to rotate the blade 160 about the entire circumference of the cornea 103, the incision in the cornea 103 forms a flap 180 that is separable from the remainder of the cornea 103 about the perimeter of the cornea 103 to expose an exposed surface 181 of the cornea 103, but remains attached at the central portion 182 of the cornea 103 as shown. Hence, the incision does not alter the optical axis 0 of the eye 102. The flap 180 can have a uniform thickness, or a varying thickness, as desired, and can have an outer diameter from about 5 mm to about 10 mm, or any other suitable dimension. The central portion can have an outer diameter of as little as about 0.5 mm or as large as 7 mm, or any other suitable dimension.

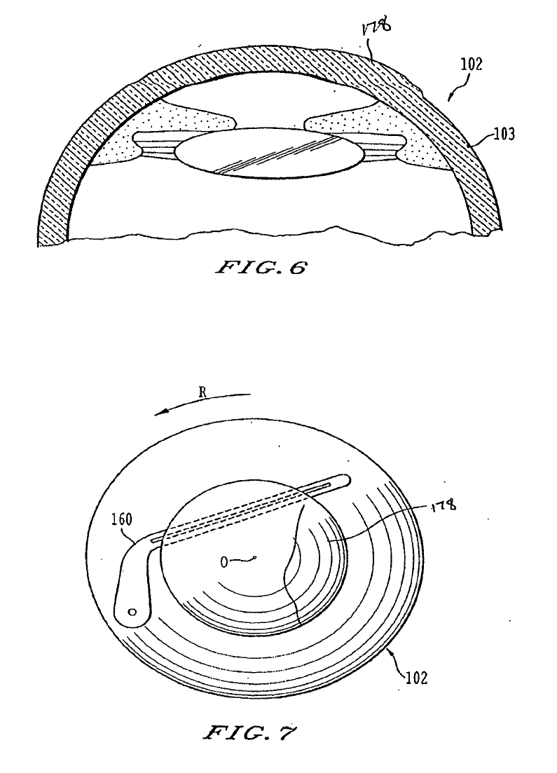

After the flap 180 has been created as described above, the suction force is discontinued, and the eye 102 can be removed from the cornea holding apparatus 104. The thickness of the exposed surface 181 can then be measured and, if appropriate, further incisions in the exposed surface 181 can be made in the manners discussed in detail below. The flap 180 can then be repositioned back onto the exposed surface 181 and the remaining portion of the cornea 103 as shown, for example, in FIG. 9, and permitted to assume a relaxed position. It is important to note that the incision forming the flap 180 relaxes the Bowman's layer of the cornea 103 to therefore change the curvature of the cornea 103 to thus correct the vision disorder in the manner described above.

The underside of the flap 180 and the exposed surface 181 of the cornea 103 can be washed with a suitable solution to remove debris from underneath the flap 180 and on the exposed surface 181. Furthermore, antibiotic drops containing an anti-infection agent can be placed on the exposed surface 181 and on the underside of the flap 180.

As indicated in FIG. 9, the edge 183 of the flap 180 can overlap a portion of the cornea 103 when the flap 180 assumes its relaxed state. In addition, if desired, an adhesive material such as syanocrylate (commonly referred to “Derma Bond” made by Ethicon Co.), or other adhesives such as polyethylene glycol hydrogels manufactured by Sharewater Polymers, Inc. or Cohesion Technologies, Inc., or Advaseal made by Focalseal, Inc., can be used to secure the flap in place during healing. Also, a short-term bandage can be attached to the front of the eye, or a punctal plug can be inserted to inhibit drainage or tear flow. The incision forming the flap 180 can then be permitted to heal for the appropriate length of time.

In addition to the process described above, further incisions or tissue shrinkage can be made in the cornea underneath the flap 180 before the flap 180 is repositioned over the exposed surface 181 to correct other vision disorders such as myopia, hyperopia or presbyopia. For example, as shown in FIG. 10, the flap 180 can be created by blade 160 of the apparatus 100 as described above, and lifted from the remaining portion of the cornea 103.

As described in more detail below, the incision creating the flap 180 can alternatively be made by a cutting tool, such as a keratome or scalpel, a razor blade, a diamond knife, a contact (fiber optic) laser, a non-contact laser having nano-second (10.sup.-9), pico-second (10.sup.-12) or femto-second (1O.sup.-15) pulses, or water-jet cutting tool as manufactured, for example, by Visijet Company. The contact or non-contact laser can emit their radiation within the infrared, visible or ultraviolet wavelength. A cutting tool such as a scalpel, a razor blade, a diamond knife, a contact (fiber optic) laser or a non-contact laser having nano-second (10.sup.-9), pico-second (10.sup.-12) or femto-second (10.sup.-15) pulses at the wavelengths described above can be used to create additional incisions 186 in the exposed surface 181. It is noted that the above lasers create the incisions 186, as well as the incision for making the flap 180, without coagulating any or substantially any of the corneal tissue. Rather, the lasers cause a series of micro explosions to occur in the cornea 103, which create the incision without any coagulation. The flap 180 can then be allowed to relax back upon the exposed surface 181 and the remainder of the cornea 103 to assume a curvature as modified by the incisions 186. The other steps of washing the flap 180 and exposed surface 181, as well as applying the antibiotic drops and so on, can then be performed as described above.

The depths of the additional incisions 186 made under the flap 180 can have dimensions sufficient the correct the degree of hyperopia or presbyopia that is being experienced by the eye. In addition, the cutting blade that can be used to form the additional incision 186 underneath the flap 180 can be flexible so that it bows when force is applied to therefore create the incision 186 as a curved incision in the cornea underneath the flap 180. Furthermore, this additional incision or incisions can be made in the underside of the flap portion 180, if desired.

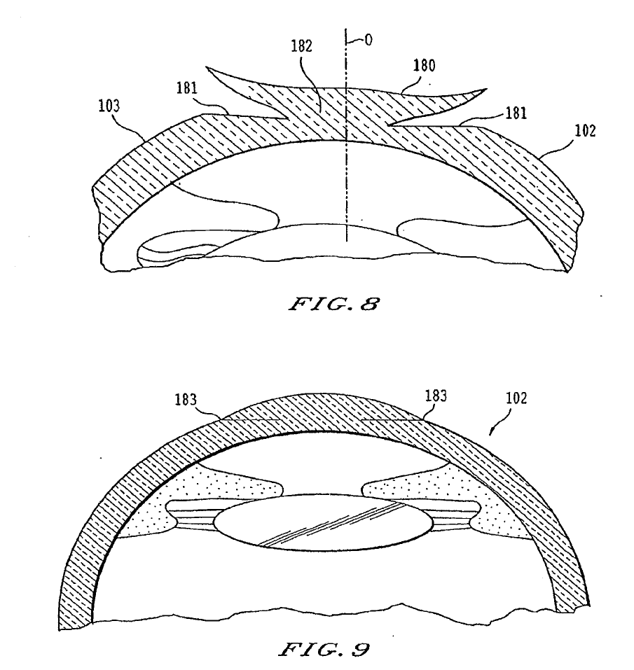

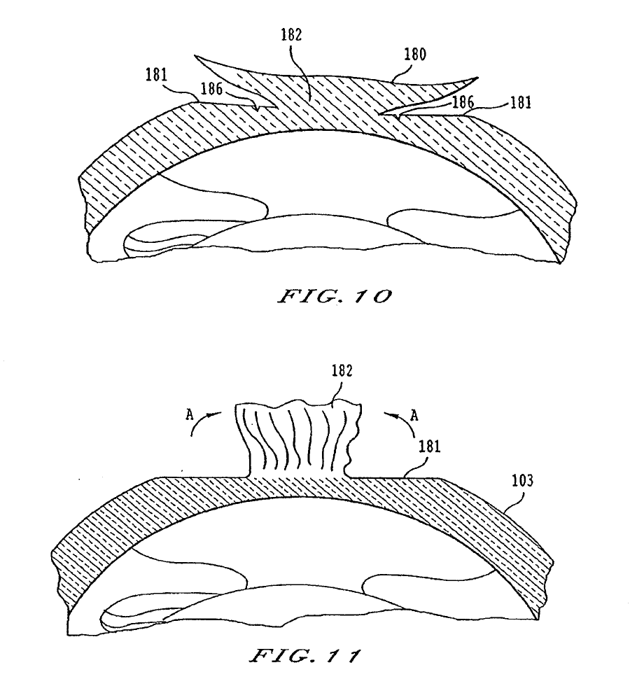

It is also noted that the cutting tools described above for making incision 186 can be used to create other types of incisions underneath the flap 180. For example, as shown in FIG. 11, the flap 180 can be lifted to expose most or all of the exposed surface 181. As shown in FIG. 12, one or more radial incisions 188 can be made in the surface of the cornea 103 underneath the flap 180 to correct for vision disorders such as myopia. It is noted that the radial incisions 188 are made without removing any or substantially any of the tissue from the exposed surface 181. Furthermore, as shown in FIG. 13, one or more radial incisions 188 can be made in the cornea underneath the flap 180, along with one or more actuate incisions 190, which correct astigmatism. As with the radial incisions 188, the actuate incisions 190 are formed without removing any or substantially any tissue from the exposed surface 181. Also, the lengths and depths of the radial and actuate incisions can vary as necessary to correct the degree of the vision disorder, and can be as deep as 95% of the remaining cornea 103.

As further shown in FIG. 13, tissue shrinkage areas 192 can be produced on the exposed surface 181 using tools such as a diathermy device, microwave emitting device, or a laser such as a contact (fiber optic) laser or a non-contact laser having nano-second (10.sup.-9), pico-second (10.sup.-12) or femto-second (1O.sup.-15) pulses. It is noted that these devices create the shrinkage areas 192 without causing any or substantially any ablation of the tissue, and without removing any or substantially any of the tissue. The shrinkage areas 192 can be circular, oval, or any other suitable shape to correct the vision disorder. It is noted that generally, radial incisions 188, such as those shown in FIG. 11 are formed to correct myopia, while actuate incisions 190 such as those shown in FIG. 12 are formed to correct astigmatism, and the shrinkage areas 192 are generally formed to correct hyperopia or presbyopia.

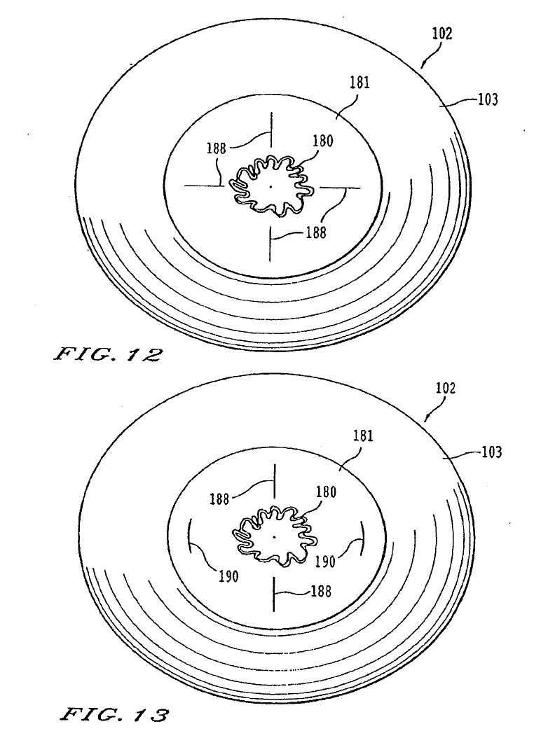

Also, as further shown in FIGS. 15 and 16, the radial incisions 188, actuate incisions 190 and shrinkage areas 192 can be made in any combination and in any amount as appropriate to correct the vision disorder. They can also be made in addition to the incisions 186 (see FIG. 10), if desired. It is noted that the shrinkage areas 192, when formed adjacent to the incisions 188 or 190, can open the incisions 188 and 190, to provide for a further correction of the myopic or astigmatic condition.

In addition, although the above discussion relates to a peripheral flap 180, the tools described above can be used to form a full flap, such as that used for the LASIK procedure as described above, or a pocket type flap as described in U.S. Pat. No. 5,964,776 cited above. The incisions 186, 188 and 190, as well as the shrinkage areas 192, can then be formed under the full flap or under the pocket type flap. Furthermore, if desired, any of the incisions or shrinkage areas can be formed in the bottom side of the flap 180, or on the bottom side of the pocket type flap or full flap, instead of or in addition to those formed on the exposed surface 181.

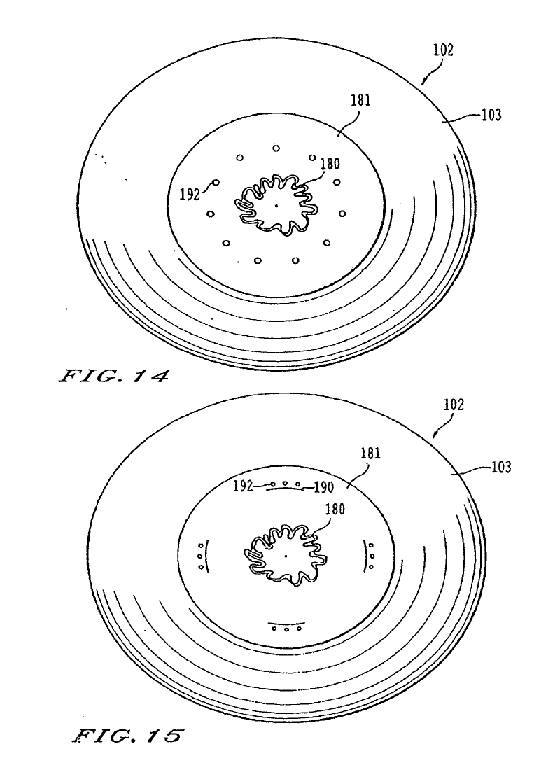

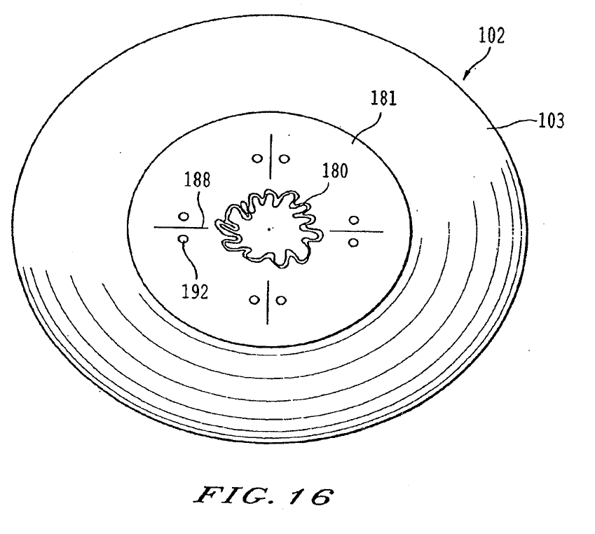

Furthermore, as shown in FIGS. 17 and 18, a laser 193, such as an Nd-YAG laser, can be used to form incisions 193 at desired depths in the stroma of the cornea 103 prior to forming the flap 180 or after forming the flap 180. The laser 193 can be a contact laser or non-contact laser pulsed at nano, pico or femto second pulses, as described above, to form the incisions 193. Also, although FIGS. 17 and 18 show the incisions 193 as being formed prior to or after creation of a peripheral flap 180, the incisions can be formed before or after a full flap, such as that used for the LASIK procedure as described above, or a pocket type flap as described in U.S. Pat. No. 5,964,776 cited above.

Although the above description is related to apparatus 100 shown in FIGS. 1 through 5, it is also noted that other tools such a water-jet or laser can be used to make the incision in the cornea 103 that forms the flap 180. For example, as shown in FIGS. 17 and 18, a cornea holding apparatus (not shown), which can be similar to cornea holding apparatus 104, can be used in conjunction with a cutting apparatus 194 such as a water-jet or a laser. Assuming, for example, that the cutting apparatus is a water-jet, a support 196 of the cutting apparatus 194 positions the cutting apparatus 194 such that the water stream from the water-jet is directed perpendicular or substantially perpendicular to the optical axis 0 of the eye 102 in a horizontal or substantially horizontal direction toward the cornea 103, so that the water stream cuts the cornea 103 tangential toward the point of contact in a manner similar to blade 160 discussed above. The support 196 can be moved manually or by a driving mechanism (not shown) along a circular track (not shown), for example, to rotate the water-jet cutting apparatus 194 about the cornea 103 along the direction R shown in FIG. 18, while keeping the water stream horizontal or substantially horizontal with respect to the surface of the cornea 103, to form a flap 180 about the circumference of the cornea 103 in a manner as described above with regard to blade 160. A guard plate 198 also can be positioned to rotate along the circular track to follow the movement of the water jet and thus block the water jet.

As explained above, the incision forming the flap 180 can be made about the entire circumference of the cornea 103, only in an astigmatic portion 178 of the cornea 103, or at any other portion of the cornea 103. The flap 180 can therefore be allowed to relax on the cornea to correct the astigmatic condition in a manner as described above. Also, additional incisions such as those described with regard to FIGS. 9 through 16 can also be made underneath the flap 180 with the appropriate tools as discussed above.

Similarly, if the cutting tool 194 is a laser, such as those described above, the supporting apparatus 196 directs the laser beam in a direction perpendicular or substantially perpendicular to the optical axis 0 of the eye, and horizontal or substantially horizontal to the cornea 103, and rotates the laser cutting tool 202 about the cornea to form a flap 180 in a manner described above. It is noted that the laser beam has an intensity and wavelength to form the incision in the cornea without coagulating or substantially coagulating the tissue of the cornea 103. Rather, the incision is formed by a series of micro explosions that occur adjacent to each other in the cornea.

It is further noted that the cutting tool can be a laser water-jet 194-1 such as that manufactured by Visijet Company can be used to create the incision for the flap 180. This type of laser water-jet, or the water jet described above, can also be used to remove the lens cortex and nucleus, to remove a clot in an artery or vein, to remove cholesterol plaque in the coronary artery, and so on.

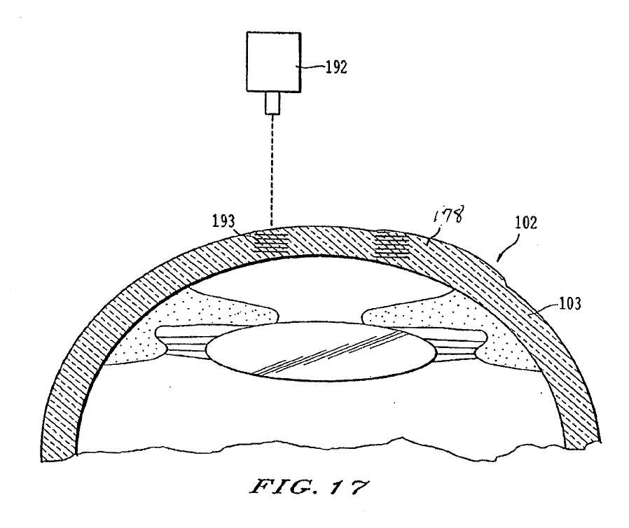

As shown in FIG. 21, the laser water-jet 194-1 includes a water-jet instrument, such as those described above, along with a fiber optic cable 200 positioned to emit laser light into the water-jet tube. The laser light can be infrared, visible, ultraviolet or any other wavelength. The water stream can act as a conduit for the laser light, so that the laser light aids in forming the incision that forms the flap 180 as described above. The water jet and laser light can be emitted from the opening 202 in the end, or from the side opening 204, or both. For example, the side opening 204 can be blocked so that the water jet and laser light only passes through end 202, or the end 202 can be blocked so that the water stream and laser light only passes out of side opening 204. In addition, the guard plate 198 include a conduit 206 can be used to remove the water, be ejected from the laser water-jet 194-1.

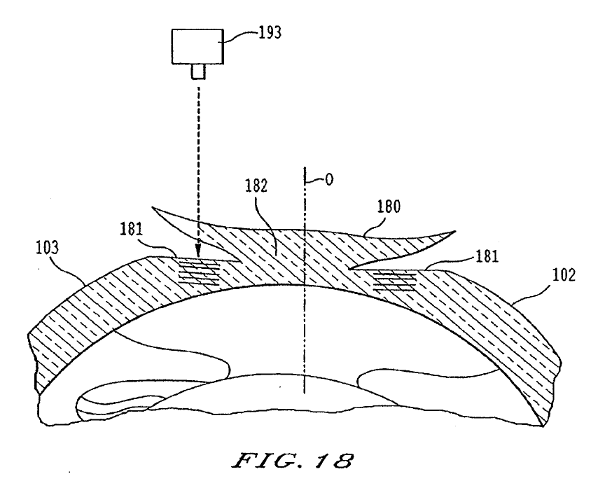

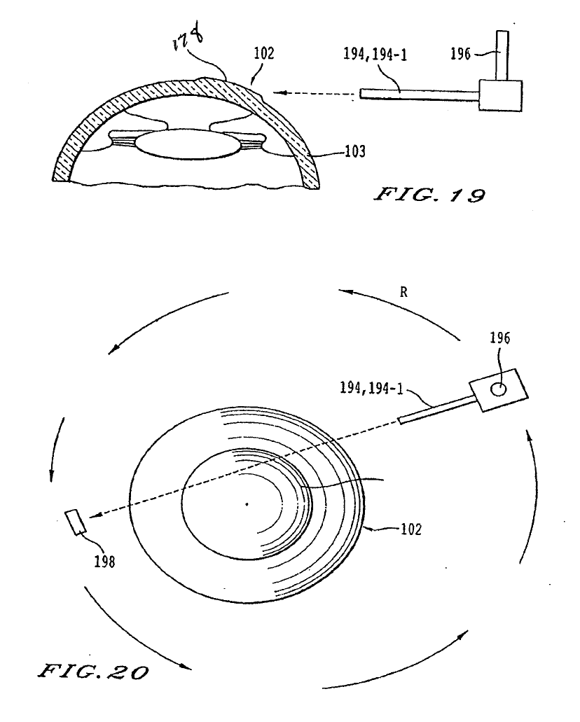

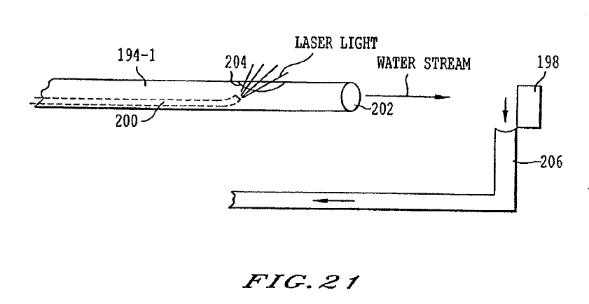

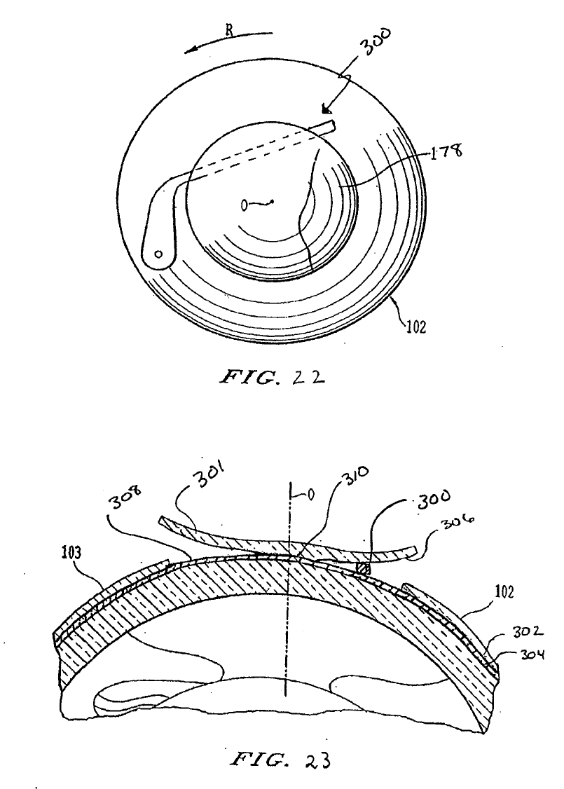

FIGS. 22-24 illustrate another embodiment of the present invention, wherein the device is configured to form a sub-epithelial flap to correct refractive error in the eye. That is, a flap 301 is formed in the epithelial layer 301 of the cornea 103. Preferably, the cutting device 300 separates the epithelial layer 302 from the Bowman's layer 304, leaving the epithelial layer attached at an area substantially surrounding the main optical axis O. However, the flap can be formed in any suitable manner and be attached to the cornea at any location of the cornea or any portion of the flap desired (or not attached to the cornea at all). For example, the flap can be attached at a periphery thereof or at a location on the cornea outside the main optical axis.

As shown in FIG. 23, the cutting device 300 preferably has a substantially rectangular or substantially square cross section. Furthermore, the cutting device is preferably a substantially thin piece of metal (such as a wire) or other material suitable for separating an epithelial layer. It is noted that the cutting device does not need to have this configuration and can have any suitable cross section (e.g., circular, oval or any other shape) and does not need to be substantially thin. The cutting device can be a blunt end of a blade or other cutting tool.

Preferably the cutting device should have suitable thickness such that it can burrow under the epithelial layer without cutting through the Bowman's layer and/or cutting into the stromal layer. Thus, conventional keratomes are inadequate as they are designed to cut into the stroma.

Cutting device 300 operates in a similar manner as the embodiments described above. First, as described above the cornea can be received in the interior surface of the cornea receiving section 108, which can include a plurality of steps or ridges (not shown) that preferably contact the surface of the live cornea 103 and assist in stabilizing the cornea from movement. As the front surface of the cornea 103 of the eye 102 is received in the receiving section 108, suction will be applied via tube 110 to the internal cavity 114 of the receiving section 108 to suck the cornea into the cavity 114.

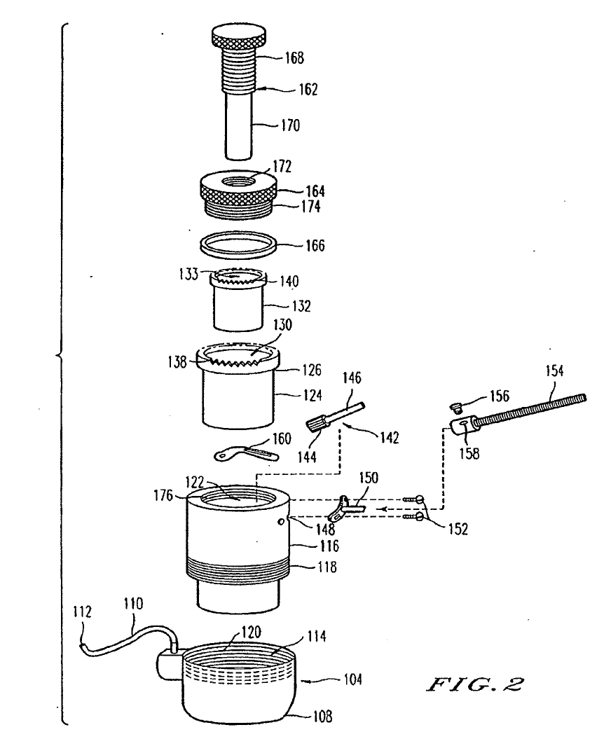

Second, as shown in FIG. 2, when the drive shaft 154 is rotated, the gear member 142 engages the teeth 138 and 142 of the large and small cylindrical members 124 and 132, respectively, and rotates the large and small cylindrical members 124 and 132. When the large and small cylindrical members 124 and 132 rotate, they move the cutting device about the circumference of the cornea 103 in a direction along arrow R to create an incision in the cornea 103, as shown in FIGS. 22 and 23. The drive shaft 154 can be rotated to cause the cutting device 300 to create an incision about the entire circumference of a cornea 103 or any portion of the circumference of the cornea 103 or in any manner desired.

Assuming, for example, that the drive shaft 154 rotates the cutting device 300 about the entire circumference of the cornea 103, the incision in the cornea 103 forms a flap 301 that is separable from the remainder of the cornea 103 about the perimeter of the cornea 103 and remains attached at the main optical axis. Moving the flap can expose surface 306 of the cornea 103. Hence, the incision does not alter the optical axis 0 of the eye 102.

It is noted that cutting device 300 can be used in any suitable flap forming device and it is not limited to the embodiments described herein.

Preferably cutting device 300 separates an internal area of the cornea offset from the main optical or visual axis 0 into first 306 and second 308 substantially ring-shaped internal surfaces. First internal corneal surface 306 faces in a posterior direction of cornea 103 and the second internal corneal surface 308 faces in an anterior direction of the cornea 103. The distance from first internal corneal surface 14 to the exterior corneal surface 28 is preferably a uniform thickness of about 5-250 microns, and more preferably about 10-50 microns, but can be any suitable thickness and does not necessarily need to be substantially uniform. A portion 310 of first and second surfaces 306 and 308 preferably remains attached to each other by an area located at the main optical axis O. The flap 301 can have a uniform thickness, or a varying thickness, as desired, and can have any suitable outer diameter. The central portion can have an outer diameter of as little as about 0.1 mm or as large as 7 mm, or any other suitable dimension.

After the flap 301 has been created as described above, the suction force is discontinued, and the eye 102 can be removed from the cornea holding apparatus 104.

The surface beneath the flap can then be ablated or altered in any manner desired, including as described above to correct refractive error in the eye.

Additionally an implant or inlay 312 can be positioned on the exposed surface, underneath the flap, as shown in FIG. 24. The inlay can be a ring or partial ring or any other suitable shape and can have any suitable refractive index. Flap 301 is lifted and pulled back, thereby exposing corneal surface 308. Preferably surface 308 is the Bowman's layer or a portion of the Bowman's layer, but layer 308 can be a portion of the epithelium or the stroma or any other suitable layer. Inlay 312 is then implanted in cornea 103 by placing inlay 312 on exposed corneal surface 308 with back surface 314 of inlay 312 resting on corneal surface 308, as seen in FIG. 24. Additionally, the lens can be altered by exposure to laser light (i.e., ablated) or in other manner desired.

Any description of the above embodiment can apply to the embodiments shown in FIGS. 22-24, unless otherwise specifically described herein.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

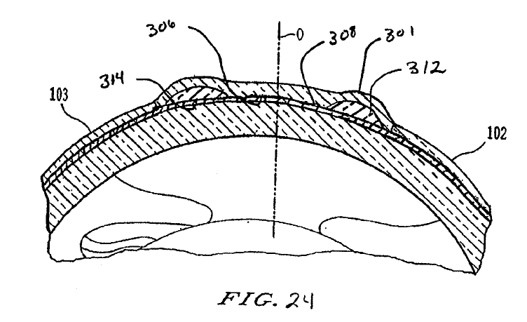

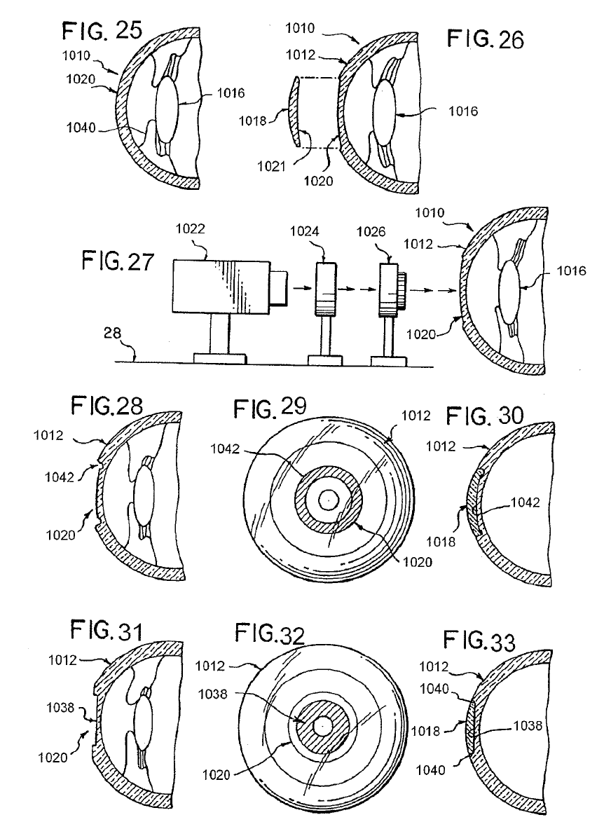

As seen in FIG. 25, an eye 1010 is shown comprising a cornea 1012, a pupil 1014, and a lens 1016. If the combination of the cornea and lens does not provide adequate vision, the cornea can be modified in accordance with the invention to modify the refractive power of the combined corneal and lens system, to thereby correct vision. This is accomplished first by removing a thin layer 1018 from the center part of a patient's live cornea 1012 by cutting via a means for removing 1019, such as a scalpel, via cutting, this thin layer being on the order of about 0.2 mm in thickness with the overall cornea being about 0.5 mm in thickness. Once the thin layer 1018 is cut and removed from the cornea, it exposes first and second opposed internal surfaces 1020 and 1021 resulting from the surgical procedure. Advantageously, it is the exposed internal surface 1020 on the remaining part of the cornea that is the target of the ablation via the excimer laser. On the other hand, the cut internal surface 1021 on the removed thin layer of the cornea can also be the target of the laser, as illustrated in FIG. 42 and discussed in further detail hereinafter.

As seen in FIG. 27, the apparatus used in accordance with the invention comprises a source of a laser beam 1022, an adjustable diaphragm 1024, and a guiding mechanism 1026, all aligned adjacent the eye 1010 and supported on a suitable base 1028.

The laser beam source 1022 is advantageously an excimer laser of the argon-fluoride or krypton-fluoride type. This type of laser will photoablate the tissue of the cornea, i.e., decompose it without burning or coagulating which would unduly damage the live tissue. This ablation removes desired portions of the cornea and thereby allows for modification of the curvature thereof.

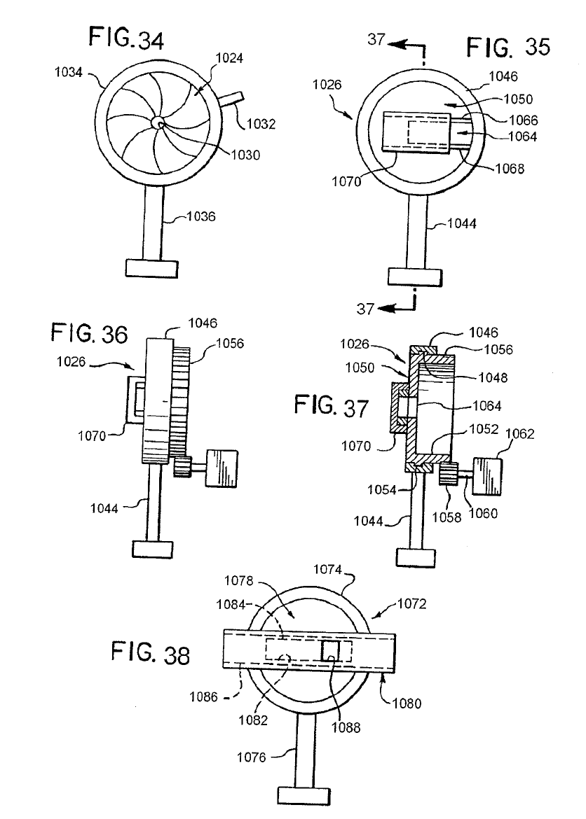

The adjustable diaphragm 1024 seen in FIGS. 27 and 34 is essentially a conventional optical diaphragm with an adjustable central orifice 1030 that can be increased or decreased in radial size by a manipulation of a lever 1032 coupled to the diaphragm. The diaphragm is advantageously supported in a ring 1034 that is in turn supported on a stand 1036 on base 1028. The material forming the diaphragm is opaque to laser light and thus when the laser is directed towards the diaphragm, it will pass therethrough only via the orifice 1030. The diaphragm 1024 can be used in conjunction with the guiding mechanism 1026, to be described in more detail hereinafter, to restrict the size of the laser beam passing to the guiding mechanism 1026, or it can be used by itself to provide ablation of the exposed internal surface 1020 of a cornea at its center.

This is illustrated in FIGS. 31-33 where a substantially disc-shaped ablated portion 1038 is formed in the central exposed internal surface 1020 by directing the laser beam 1022 through orifice 1030 of the diaphragm 1024. By modifying the size of the orifice, the disc-shaped ablated portion 1038 can be varied in size. Also, by varying the size of the orifice over time, either a concave or convex ablated portion can be formed, as desired. As shown in FIG. 1033, once the ablated portion 1038 is as desired, the previously removed thin layer 1018 is replaced onto the cornea in the ablated portion 1038 and can be connected thereto via sutures 1040.

Because the ablated portion 1038 as seen in FIG. 31 is essentially a uniform cylindrical depression in the exposed internal surface 1020, when the thin corneal layer 1018 is replaced, the curvature of the cornea is decreased, thereby modifying the refractive power of the cornea and lens system.

As seen in FIG. 34, lever 1032 is used to vary the size of orifice 1030, and is capable of being manipulated by hand or by a suitable conventional motor, which can be coordinated to provide an expansion or contraction of the orifice as necessary over time.

As seen in FIGS. 27, 35, 36 and 37, the guiding mechanism 1026 can be utilized in addition to or in place of the diaphragm 1024 to guide the laser light onto the cornea. This guiding mechanism 1026 is especially advantageous for forming an annular ablated portion 1042 in surface 1020 as seen in FIGS. 28-30 for increasing the overall curvature of the cornea.

As seen in FIGS. 28 and 29, this annular ablated portion 1042 is spaced from the center of the exposed internal surface 1020 and when the previously removed thin corneal layer 1018 is replaced and sutured, the thin layer tends to be more convex, thereby modifying the overall curvature of the cornea.

As seen in FIGS. 35-37, the guiding mechanism 1026 comprises a stand 1044 supporting a ring 1046, this ring having a radially inwardly facing recess 1048 therein. A disc 1050, which is opaque to laser light, is located inside the ring and has a cylindrical extension 1052 with an outwardly facing flange 1054 rotatably and slidably received in the recess. On the cylindrical extension 1052 which extends past ring 1046 is an exterior toothed gear 1056 that is in engagement with a pinion 1058 supported on a shaft 1060 of a motor 1062. Rotation of pinion 1058 in turn rotates gear 1056 and disc 1050.

The disc 1050 itself has an elongated rectangular orifice 1064 formed therein essentially from one radial edge and extending radially inwardly past the center point of the disc. Adjacent the top and bottom of the orifice 1064 are a pair of parallel rails 1066 and 1068 on which a masking cover 1070, which is U-shaped in cross section, is slidably positioned. Thus, by moving the masking cover 1070 along the rails, more or less of the orifice 1064 is exposed to thereby allow more or less laser light to pass therethrough and onto the cornea. Clearly, the larger the orifice, the larger the width of the annular ablated portion 1042 will be. By rotating the disc, the orifice 1064 also rotates and thus the annular ablated portion 1042 is formed.

### Embodiment of FIG. 38

Referring now to FIG. 38, a modified guiding mechanism 1072 is shown which is similar to guiding mechanism 1026 shown in FIGS. 35-37 except that the size of the orifice is not variable. Thus, the modified guiding mechanism 1072 is comprised of a ring 1074 on a stand 1076, an opaque disc 1078 which is rotatable in the ring via a suitable motor, not shown, and a slidable masking cover 1080. Disc 1078 has a rectangular orifice 1082 extending diametrically there across with parallel rails 1084 and 1086 on top and bottom for slidably receiving the masking cover 1080 thereon, this cover being U-shaped for engagement with the rails. The masking cover 1080 has its own orifice 1088 therein which aligns with orifice 1082 on the disc. Thus, by sliding the masking cover 1080 along the rails of the disc, the location of the intersection of orifice 1088 and orifice 1082 can be varied to vary the radial position of the overall through orifice formed by the combination of these two orifices. As in guiding mechanism 1026, the masking cover 1080 and disc 1078 are otherwise opaque to laser light except for the orifices.

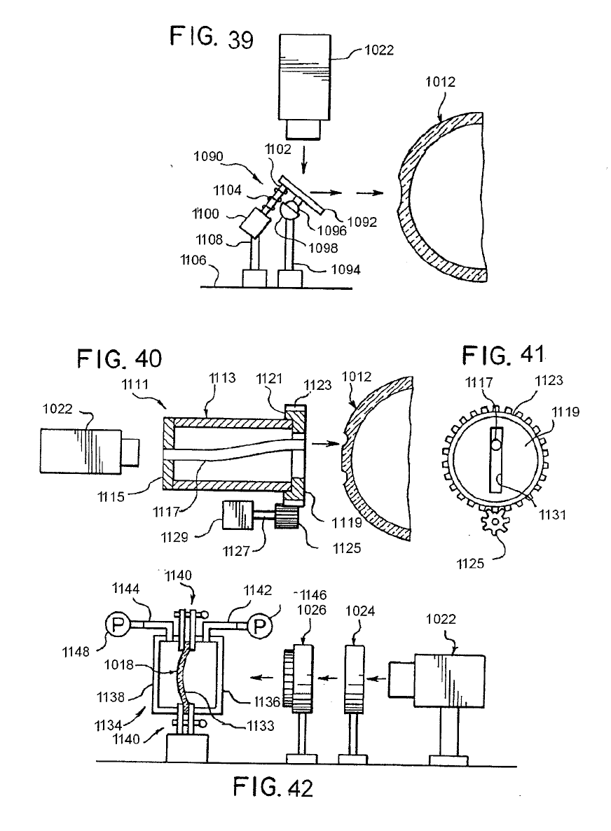

### Embodiment of FIG. 39

Referring now to FIG. 39, a second modified guiding mechanism 1090 is shown for directing laser light from laser beam source 1022 to the cornea 1012 along the desired predetermined pattern. This guiding mechanism 1090 comprises a mirror 1092 universally supported on a stand 1094 via, for example, a ball 1096 and socket 1098 joint. This mirror 1092 can be pivoted relative to the stand through the universal joint by means of any suitable devices, such as two small piezoelectric motors which engage the mirror at 90° intervals. For example, such a piezoelectric motor 1100 having a plunger 1102 coupled thereto and engaging the rear of the mirror can be utilized with a spring 1104 surrounding the plunger and maintaining the mirror in a null position. The motor 1100 is rigidly coupled to a base 1106 via a stand 1108. The second piezoelectric motor, not shown, can be located so that its plunger engages the rear of the mirror 90° from the location of motor 1100. By using these two motors, springs and plungers, the mirror 1092 can be fully rotated in its universal joint to direct the laser beam from source 1022 onto the cornea 1012 to ablate the cornea in a predetermined pattern.

### Embodiment of FIGS. 40-41

Referring now to FIGS. 40 and 41, a third modified guiding mechanism 1111 is shown for ablating a cornea 1012 via directing laser light from laser source 1022. This modified guiding mechanism 1111 basically comprises a cylindrical housing 1113 having an opaque first end 1115 rotatably receiving the end of a fiber optic cable 1117 therein. The second end 1119 of the housing comprises a rotatable opaque disc having a flange 1121 engaging the housing and an external gear 1123 which in turn engages pinion 1125, which is driven via shaft 1127 and motor 1129. Thus, rotation of the pinion results in rotation of gear 1123 and thus the opaque second end 1119 of the housing. This second end 1119 has a diametrically oriented rectangular orifice 1131 therein which receives the other end of the fiber optic cable 1117 therein. That end of the fiber optic cable is either dimensioned so that it fits fairly tightly into the orifice or there is an additional suitable assembly utilized for maintaining the fiber optic cable end in a predetermined position in the orifice during rotation of the second end. However, this end would be movable radially of the orifice to change the position of the annular ablated portion formed by utilizing this guiding mechanism.

### Embodiment of FIG. 42

Referring now to FIG. 42, rather than ablating the exposed internal surface 1020 on the cornea 1012, the inner surface 10133 of the removed thin corneal layer 1018 can be ablated utilizing the apparatus shown in FIG. 42. Likewise, the apparatus of FIG. 42 can be used on an eye bank cornea removed from the eye and then positioned in the patient's eye to modify the curvature of the patient's combined corneal structure. This apparatus as before includes the source of the laser light 1022, an adjustable diaphragm 1024, and a guiding mechanism 1026. In addition, an assembly 1134 is utilized to support the rather flimsy removed thin corneal layer. This assembly 1134 comprises a pair of laser light transparent cups 1136 and 1138 that are joined together in a sealing relationship via clamps 1140 and engage therebetween the outer periphery of the thin corneal layer 1018. Each of the cups has an inlet pipe 1142, 1144 for injecting pressurized air or suitable fluid into each via pumps 1146 and 1148. By using this pressurized container, the thin corneal layer 1018 is maintained in the desired curvature so that the laser beam can provide a precise ablated predetermined pattern therein. In order to maintain the curvature shown in FIG. 42, the pressure on the right hand side of the thin layer is slightly greater than that on the left hand side.

Once the thin corneal layer 1018 is suitably ablated as desired, it is replaced on the exposed internal surface 1020 of the cornea and varies the curvature of the overall cornea as described above and illustrated in FIGS. 28-33.

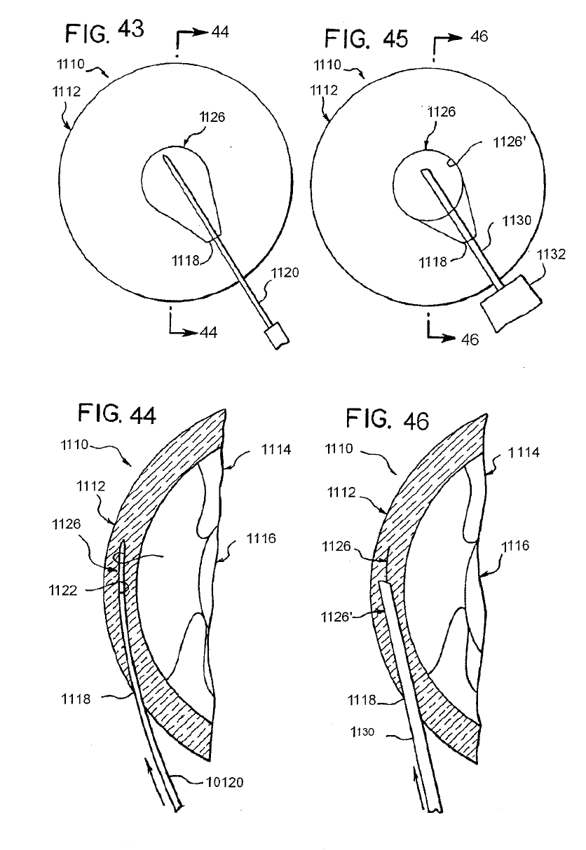

### Embodiment of FIGS. 43-51

Referring now to FIGS. 43-51, a patient's live in situ eye 1110 is shown for the treatment of myopia in accordance with the present invention. Eye 1110 includes a cornea 1112, a pupil 1114, and a lens 1116, and is treated in accordance with the present invention without freezing the cornea.

Correction of myopia can be achieved by decreasing the curvature of the outer surface of cornea 1112 (i.e., flattening the central portion of the cornea). This is accomplished by first cutting an incision 1118 into the epithelium of cornea 1112. Incision 1118 may be curved or straight, and is preferably about 2.0-3.0 mm long and about 3.0-6.0 mm away from the center of cornea 1112. A laser or spatula (i.e., a double-edge knife) may be used to make incision 1118 in cornea 1112.

As seen in FIGS. 43 and 44, once incision 1118 is made, a spatula 1120 is inserted into incision 1118 to separate an internal area of live cornea 1112 into first and second opposed internal surfaces 1122 and 1124, thereby creating an intrastromal or internal pocket 1126. First internal surface 1122 faces in the posterior direction of eye 1110, while second internal surface 1124 faces in the anterior direction of eye 1110, and both of these surfaces extend radially relative to the center of the cornea.

As seen in FIGS. 43 and 44, pocket 1126 is created by moving spatula 1120 back and forth within an intrastromal area of cornea 1112. It is important when creating pocket 1126 to keep spatula 1120 in substantially a single plane and substantially tangential to the cornea's internal surfaces to prevent intersecting and rupturing the descemet or Bowman's membrane.

Preferably, spatula 1120 is about 3.0-12.0 mm long with a thickness of about 0.1-1.0 mm, and a width of about 0.1-1.2 mm. Spatula 1120 may be slightly curved, as seen in FIG. 44, or may be straight.

While a spatula 1120 is shown in FIGS. 43 and 45 for separating the internal surfaces of cornea 1112, a fiber optic cable coupled to a laser beam source may be used instead of spatula 1120 to separate cornea 1112 into first and second opposed internal surfaces 1122 and 1124.

As seen in FIGS. 45 and 46, after pocket 1126 is formed, a fiber optic cable tip 1130 coupled to a fiber optic cable 1132, which is in turn coupled to a laser, is then inserted through incision 1118 and into pocket 1126 for ablating a substantially circular area of cornea 1112, thereby removing a substantially disc-shaped portion of cornea 1112 to form a disc-shaped cavity 1126′. The laser beam emitted from tip 1130 may be directed upon either first internal surface 1122, second internal surface 1124, or both, and removes three-dimensional portions therefrom via ablation. The fiber optic cable can be solid or hollow as desired.

The laser source for fiber optic cable 1132 is advantageously a long wavelength, infrared laser, such as a CO 2, an erbium or holmium laser, or a short wavelength, UV-excimer laser of the argon-fluoride or krypton-fluoride type. This type of laser will photoablate the intrastromal tissue of the cornea, i.e., decompose it without burning or coagulating.

FIGS. 49-51 illustrate three different configurations of the tip of a fiber optic cable for ablating the cornea. In FIG. 49, tip 1130 has a substantially straight end for directing the laser beam parallel to the tip. As seen in FIG. 50, tip 1130′ has an end with an angled surface for directing the laser beam at an acute angle of preferably 450 relative to the tip to aid in ablating the cornea as desired. In FIG. 51, tip 1130′ has a curved end for bending the laser beam to aid ablating the cornea as desired.

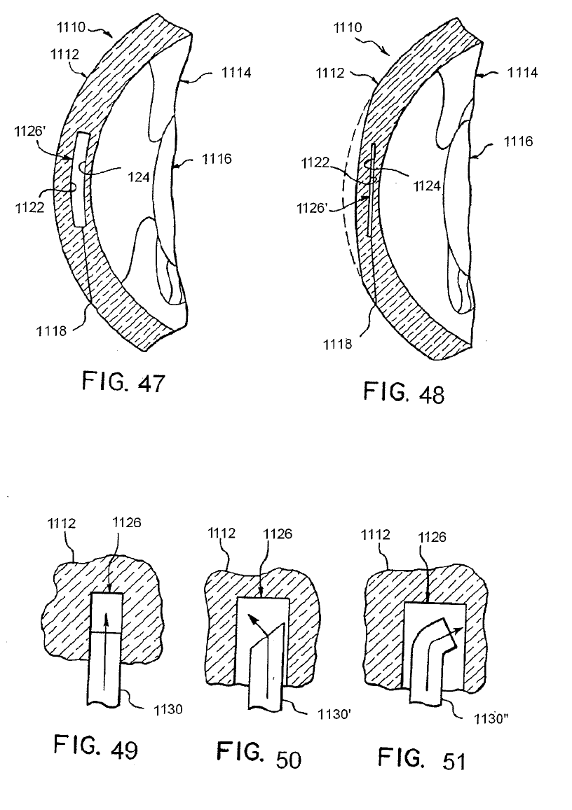

As seen in FIG. 47, cornea 1112 is shown with the substantially disc-shaped cavity 1126′ formed at the center of cornea 1112 just after tip 1130 has been removed and prior to cornea 1112 collapsing or flattening. The disc-shaped cavity 1126′ can be varied in size and shape, depending upon the amount of curvature modification needed to correct the patient's eyesight. Accordingly, any three-dimensional intrastromal area of the cornea may be removed to modify the cornea as desired. The intrastromal area removed can be uniform or non-uniform. For example, more material can be removed from the periphery of the cornea than from the center portion. Alternatively, more material can be removed from the center portion than from the peripheral area. The removal of peripheral portions of the cornea result in an increase of the curvature of the center portion of the cornea after the collapse of the peripheral area.

As seen in FIG. 48, after pocket 1126 is ablated and tip 1130 removed, the ablated cavity 1126′ then collapses under normal eye pressure to recombine ablated first and second internal surfaces 1122 and 1124 together. This collapsing and recombining of the intrastromal area of the cornea decreases the curvature of the central portion of cornea 1112 from its original shape shown in broken lines to its new shape as seen in FIG. 48. After a period of time, depending on the patient's healing abilities, the ablated surfaces heal and grow back together, resulting in a permanent modification of the corneals curvature.

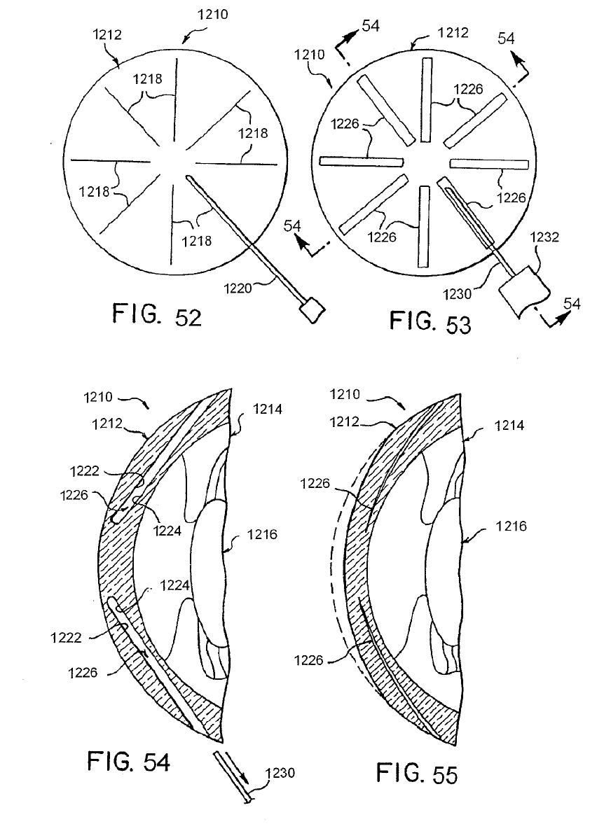

### Embodiment of FIGS. 52-55

Referring now to FIGS. 52-55, an eye 1210 is shown for the treatment of myopia in accordance with another embodiment of the present invention, and includes a cornea 1212, a pupil 1214, and a lens 1216, the cornea being treated without freezing it. In this embodiment, correction of myopia is accomplished by first making a plurality of radially directed intrastromal incisions 1218 with a flat pin or blade spatula 1220. These incisions 1218 separate the cornea 1218 into first and second opposed internal surfaces 1222 and 1224 at each of the incisions 1218. First internal surfaces 1222 face in the posterior direction of eye 1210, while second internal surfaces 1224 face in the anterior direction of eye 1210, and both extend radially relative to the center of the cornea. Spatula 1220 may have a straight or curved blade with a maximum diameter of about 0.1-0.2 mm. A laser may be used instead of spatula 1220 to make incisions 1218, if desired.

Incisions or unablated tunnels 1218 extend generally radially towards the center of cornea 1212 from its periphery. Preferably, incisions 1218 stop about 3.0 mm from the center of cornea 1212, although incisions 1218 may extend to the center of cornea 1212, depending upon the degree of myopia. Incisions 1218 will normally extend about 3.0-10.0 mm in length, again depending on the amount of change desired in curvature of cornea 1112. While only radial incisions have been shown, it will be apparent to those skilled in the art that the incisions may be non-radial, curved, or other shapes. When creating incisions 1218, it is important to keep the spatula 1220 in substantially a single plane so as not to intersect and puncture the descemet or Bowman's membrane.

Once intrastromal incisions 1218 have been created with spatula 1220, a fiber optic cable tip 1230 coupled to a fiber optic cable 1232 and a laser is then inserted into each of the incisions 1218 for ablating tunnels 1226 to the desired size. The laser beam emitted from tip 1230 may be directed upon either first internal surface 1222, second internal surface 1224, or both for ablating tunnels 1226 and removing three-dimensional portions from these surfaces.

The laser source for cable 1232 is advantageously similar to the laser source for cable 1132 discussed above.

Referring now to FIGS. 54 and 55, a pair of ablated tunnels 1226 are shown. In FIG. 54, cornea 1212 is shown with ablated tunnels 1226 just after tip 1230 has been removed and prior to tunnels 1226 collapsing or flattening. In FIG. 55, cornea 1212 is shown after ablated tunnels 1226 have collapsed to recombine first and second internal surfaces 1222 and 1224, thereby flattening cornea 1212. In other words, this collapsing and recombining of the intrastromal area of the cornea decreases the curvature of the central portion of cornea 1212 from its original shape shown in broken lines to its new shape as seen in FIG. 55. By collapsing intrastromal tunnels, this allows the outer surface of the cornea to relax, i.e., decrease surface tension, thereby permitting flattening of the cornea.

### Embodiment of FIGS. 56-59

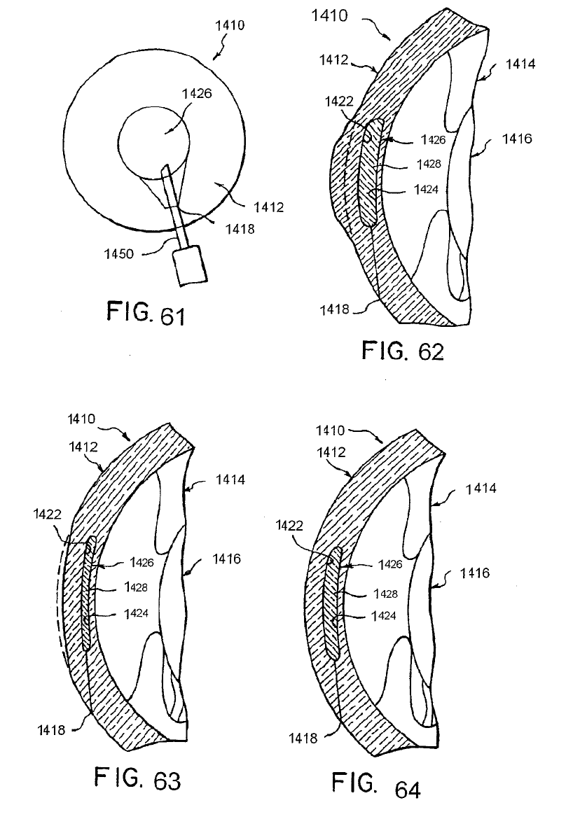

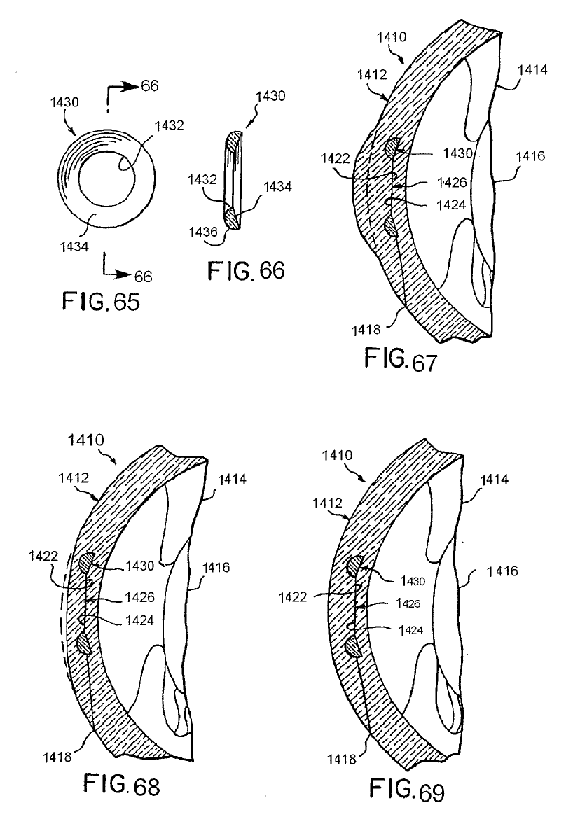

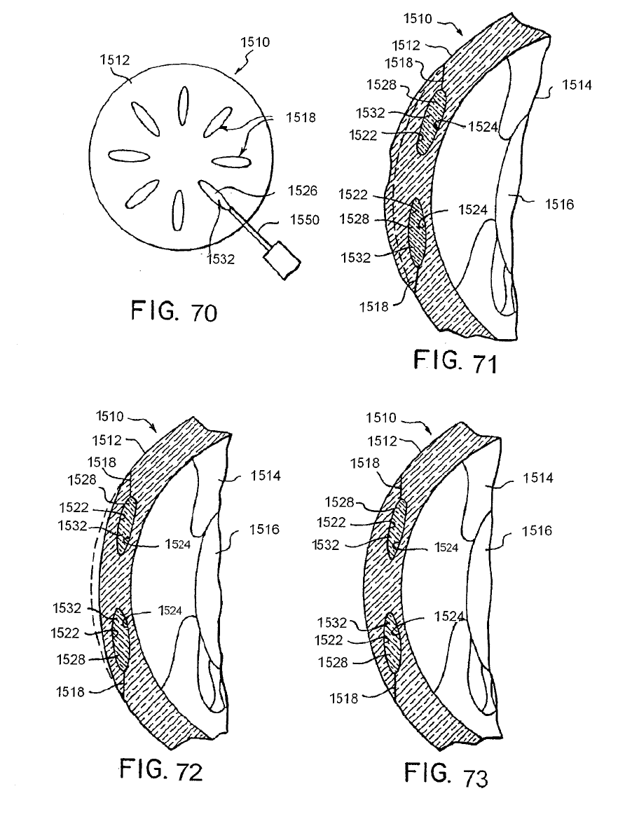

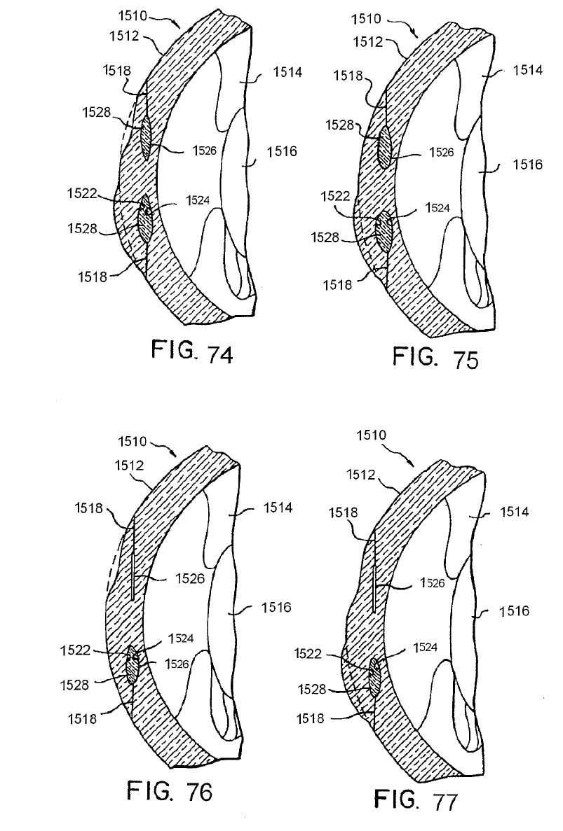

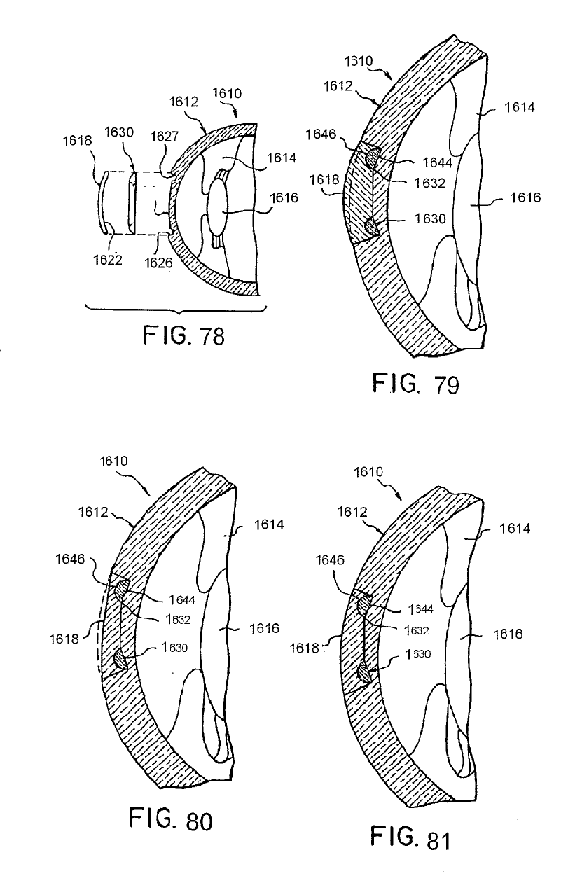

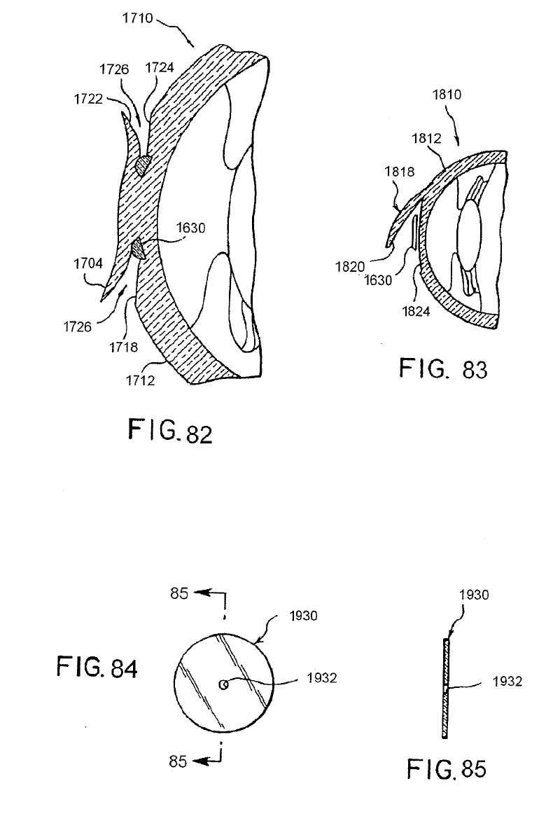

Referring now to FIGS. 59-59, an eye 1310 is shown for the treatment of hyperopia in accordance with another embodiment of the present invention. Eye 1310 includes a cornea 1312, a pupil 1314, and a lens 1316. Correction of hyperopia can be achieved by increasing the curvature of the outer surface of cornea 1312 (i.e., making the central portion of the cornea more curved), without freezing the cornea.

This is accomplished by making a plurality of intrastromal incisions or tunnels 1318 with a spatula 1320 to form first and second opposed internal surfaces 1322 and 1324. Tunnels 1318 extend substantially radially towards the center of cornea 1312. While eight equally spaced, radial tunnels 1318 are shown, it will be apparent to those skilled in the art that more or fewer tunnels with varying distances apart may be made, depending upon the amount of curvature modification needed.

The initial step of making incisions or tunnels 1318 of FIGS. 56-59 is similar to the initial step of making incisions 1218 of FIGS. 52-55. Accordingly, spatula 1320 is similar to spatula 1220 discussed above. Likewise, a laser may be used to make incisions or tunnels 1318 instead of spatula 1320.

Once tunnels 1318 are created, a fiber optic cable tip 1330 extending from fiber optic cable 1332 is inserted into each tunnel 1318 to direct a laser beam on either first internal surface 1322, second internal surface 1324, or both internal surfaces to coagulate an intrastromal portion of cornea 1312. As seen in FIG. 58, a point 1326 at the end of each of the tunnels 1318 is coagulated. Preferably, coagulation points 1326 lie substantially on the circumference of a circle concentric with the center of cornea 1312. The size of the circle forming coagulation points 1326 depends upon the amount of curvature modification needed. Likewise, the number of coagulation points and their positions in the cornea depend upon the desired curvature modification needed.

Coagulating intrastromal points of the cornea 1312, such as coagulation points 1326, with a laser causes those points of the cornea, and especially the collagen therein, to heat up and shrink. This localized shrinkage of the intrastromal portion of the cornea causes the outer surface of the cornea to be tightened or pulled in a posterior direction at each of the coagulation points, and thereby causes an increase in the overall curvature of the cornea as seen in FIG. 59. Coagulation, rather than ablation, is accomplished by using a laser having a wavelength which essentially cooks the corneal tissue and which is between the wavelengths associated with long infrared light and short ultraviolet light.

### Embodiment of FIG. 60

As seen in FIG. 60, rather than using a laser to remove corneal tissue in the cavities 1126 formed in the cornea 1112 or to form those cavities, a rotating drill tip 1400 suitably coupled to a rotary or oscillating power source can be used to ablate the tissue by cutting. Likewise, any other suitable mechanical device can be used to remove the corneal tissue or form the cavities. A suitable evacuation device, such as a vacuum tube, can also be used to aid in evacuating from the cavity the tissue removed from the cornea.

### Embodiment of FIGS. 61-69

Referring now to FIGS. 61-69, a patient's live in situ eye 1410 is shown for the treatment of hyperopia or myopia and/or improving a patient's vision by removing opaque portions of the cornea in accordance with the present invention. The eye 1410 of FIGS. 61-64 and 67-69 includes a cornea 1412, a pupil 1414 and a lens 1416, and is treated in accordance with the present invention without freezing any portion of cornea 1412.

Correction of myopia and hyperopia can be achieved by modifying the curvature of the outer surface of cornea 1412, i.e., flattening the central portion of a cornea in the case of myopia or increasing the curvature in the case of hyperopia. This is accomplished by first cutting an incision 1418 into the epithelium of cornea 1412 as seen in FIG. 61. Incision 1418 may be curved or straight, and is preferably about 2.0-3.0 mm long and about 3.0-6.0 mm away from the center of cornea 1412. A laser or a doubleedge knife may be used to make incision 1418 in cornea 1412.

As seen in FIGS. 61-64 and 67-69, once incision 1418 is made, a spatula or laser probe is inserted into incision 1418 to separate an internal area of live cornea 1412 into first and second opposed internal surfaces 1422 and 1424, thereby creating an intrastromal or internal pocket 1426 as in the previous embodiment of FIGS. 43-51. First internal surface 1422 faces in the posterior direction of eye 1410, while second internal surface 1424 faces in the anterior direction of eye 1410, and both of these surfaces extend radially relative to the center of the cornea 1412.

Pocket 1426 can have corneal tissue removed from either or both of internal surfaces 1422 and 1424. In other words, internal surfaces 1422 and 1424 of intrastromal pocket 1426 can be ablated or cut to define a cavity. The ablating or removing of the internal surfaces 1422 and 1424 of cornea 1412 is particularly desirable to remove opaque areas of cornea 1412. Alternatively, the internal surfaces 1422 and 1424 of cornea 1412 can be removed by a scalpel or a diamond tipped drill similar to the embodiments discussed above. Pocket 1426 can be created by substantially the same method as previously discussed. Of course, incision 1418 and pocket 1426 can be made in one single step by a laser or a cutting mechanism. Alternatively, none of the corneal tissue can be removed from internal surfaces 1422 and 1424.

As shown in FIGS. 61-64 and 67-69, once the pocket 1426 is formed, an ocular material 1428 or 1430 is inserted into pocket 1426 by a tool 1450. Ocular material 1428 or 1430 as used herein refers to transparent fluids or solids or any combination thereof. In the examples of FIGS. 62-64, the ocular material is a gel or fluid type material 1428, which can be injected into pocket 1426 via tool 1450. In other words, in the examples of FIGS. 62-64, tool 1450 is a needle for injecting ocular material 1428 into pocket 1426. In examples of FIGS. 67-69, the ocular material is a flexible, resilient ring shaped member 1430.

In either case, ocular material 1428 or 1430 can have either the same refractive index as the intrastromal tissue of cornea 1412 or a different refractive index from the intrastromal tissue of cornea 1412. Thus, the vision of the patient can be modified by curvature modification and/or by changing the refractive index. Moreover, the patient's vision can be modified by merely removing opaque portions of the cornea and replacing them with ocular material with a refractive index the same as the intrastromal tissue of cornea 1412.

In the examples of FIGS. 62-64 using ocular material 1428, pocket 1426 can be overfilled, partially filled, or completely filled to modify the cornea as needed. The cavity of pocket 1426 can be filled completely with the ocular material to restore the normal curvature of cornea 1426 as seen in FIG. 64. The amount of ocular material introduced to pocket 1426 can be increased to increase the curvature of the cornea from the original curvature to treat hyperopia as seen in FIG. 62. Alternatively, the amount of the ocular material introduced to pocket 1426 can be reduced to decrease the curvature or flatten cornea 1412 from the original curvature to treat myopia as seen in FIG. 63. This method is suitable for correctly vision of 12 diopters or more. After the pocket 1426 is filled, the internal surfaces 1422 and 1424 of pocket 1426 come together to encapsulate ocular material 1428 within cornea 1412. The surfaces heal and grow back together, resulting in a permanent modification of the corneals curvature.

The ocular material 1428 injected into pocket 1426 can be any suitable material that is bio-compatible and does not visually interfere with the patient's eyesight. Preferably, the ocular material 1428 of FIGS. 62-64 is a transparent gellable collagen such as gelatin in an injectable form which is available from various commercial sources as known in the art. Generally, the collagen to be used in the present invention is a type I collagen. Of course, ocular material 1428 can be a transparent or translucent bio-compatible polymer gel such as a silicone gel or an injectable polymethylmethacrylate. Preferably, ocular material 1428 is a polymeric material that is transparent, flexible, and hydrophilic. It will be understood by those skilled in the art from this disclosure that ocular material 1428 can be any suitable polymeric material. Of course, ocular material 1428 can be a flexible solid or semi-solid material as shown in the examples of FIGS. 65-69 discussed below regarding ocular material 1430 which can be made from collagen or synthetic polymers such as acrylic polymers, silicones and polymethylmethacrylates.

Referring now to the examples of FIGS. 67-69 using a solid or semi-solid ocular material or implant 1430, tool 1450 is utilized to insert ocular material or implant 1430 through the small opening formed by incision 1418 in the external surface of cornea 1412, as seen in FIG. 61 so that ocular material or implant 1430 can be implanted into pocket 1426 and centered about the main optical axis of eye 1410. Ocular material or implant 1430 is preferably a resilient, flexible member, which can be folded for insertion into pocket 1426 through the small opening formed by incision 1418.

The ocular implant 1430 is made from a bio-compatible transparent material. Preferably, ocular implant 1430 is made from any suitable transparent polymeric material. Suitable materials include, for example, collagen, silicone, polymethylmethacrylate, acrylic polymers, copolymers of methyl methacrylate with siloxanylalkyl methylacrylates, cellulose acetate butyrate and the like. Such materials are commercially available from contact lens manufacturers. For example, optical grade silicones are available from Allergan, Alcon, Staar, Chiron and bolab. Optical grade acrylics are available from Allergan and Alcon. A hydrogel lens material consisting of a hydrogel optic and polymethylmethacrylate is available from Staar.

Similar to the fluid type ocular material 1428, discussed above, solid or semi-solid ocular material or implant 1430 can overfill, partial fill or completely fill pocket 1426 to modify cornea 1412 as needed. While ablation or removal of intrastromal tissue of pocket 1426 is required for decreasing the curvature of cornea 1412 as seen in FIG. 68, or for maintaining the original curvature of cornea 1412 as seen in FIG. 69, such ablation or removal of intrastromal tissue of pocket 1426 is not necessary for increasing the curvature of cornea 1412. In any event, the amount of intrastromal tissue to be removed, if any, from pocket 1426 depends on the shape of ocular material 1430 and the desired resultant shape of cornea 1412.

As seen in FIGS. 65 and 66, ocular material or implant 1430 has a substantially annular ring shape with a center opening or circular hole 1432. Center opening 1432 allows intrastromal fluids to pass through ocular material or implant 1430. Preferably, ocular material 1430 has a circular periphery with an outer diameter in the range of about 3.0 mm to about 9.0 mm. Center opening 1432 preferably ranges from about 1.0 mm to about 8.0 mm. The thickness of ocular material 1430 is preferably about 20 microns to about 1000 microns.

In the embodiment of FIGS. 65-69, ocular material or implant 1430 has a planar face 1434 and a curved face 1436. Planar face 1434 forms a frustoconically shaped surface, which faces inwardly towards the center of eye 1410 in a posterior direction of eye to contact internal surface 1424 of pocket 1426. Curved face 1436 can be shaped to form a corrective lens or shaped to modify the curvature cornea 1412 as seen in FIGS. 67 and 68. Of course, ocular material 1430 can be shaped to replace opaque areas of cornea 1412, which have been previously removed, and/or to form a corrective lens without changing the curvature of cornea 1412 as seen in FIG. 69.

When center opening 1432 is about 2.0 mm or smaller, center opening 1432 acts as a pin hole such that the light passing through is always properly focused. Accordingly, ocular material 1430 with such a small center opening 1432 can be a corrective lens, which is not severely affected by center opening 1432. However, when ocular material 1430 has its center opening 1432 greater than about 2.0 mm, then ocular material 430 most likely will have the same refractive index as the intrastromal tissue of cornea 1412 for modifying the shape of cornea 1412 and/or replacing opaque areas of the intrastromal tissue of cornea 1412. Of course, all or portions of ocular material 1430 can have a refractive index different from the intrastromal tissue of cornea 1412 to correct astigmatisms or the like, when center opening 1432 is greater than about 2.0 mm.

The amount of curvature modification and/or the corrective power produced by ocular material 1430 can be varied by changing the thickness, the shape, the outer diameter and/or the size of the center opening 1432. Moreover, instead of using a continuous, uniform ring as illustrated in FIGS. 65 and 66, ocular material 1430 can be a ring with non-uniform cross-section in selected areas as necessary to correct the patient's vision. In addition, ocular material 1430 could be replaced with a plurality of separate solid or semi-solid ocular implants at selected locations within pocket 1426 of cornea 1412.

### Embodiment of FIGS. 70-77

Referring now to FIGS. 70-77, an eye 1510 is shown for the treatment of hyperopia or myopia and/or improving vision by removing opaque portions of the cornea, in accordance with another embodiment of the present invention. Eye 1510 includes a cornea 1512, a pupil 1514, and a lens 1516. As in the previous embodiments, cornea 1512 is treated without freezing it.

In this embodiment, correction of hyperopia or myopia or removal of opaque portions can be accomplished by first making a plurality of radially directed intrastromal incisions 1518 with a flat pin, laser or blade spatula similar to the procedure mentioned above discussing the embodiment of FIGS. 52-55. These incisions 1518 separate cornea 1512 into first and second opposed internal surfaces 1522 and 1524, respectively, at each of the incisions 1518. First internal surfaces 1522 face in the posterior direction of eye 1510, while second internal surfaces 1524 face in the anterior direction of eye 1510, and both extend radially relative to the center of cornea 1512.

Incisions or unablated tunnels 1518 extend generally radially towards the center of cornea 1512 from its periphery. Preferably, incisions 1518 stop about 3.0 mm from the center of cornea 1512, although incisions 1518 may extend to the center of cornea 1512, depending upon the degree of hyperopia or myopia. Incisions 1518 will normally extend about 3.0-10.0 mm in length, again depending on the amount of change desired in curvature of cornea 1512. While only radial incisions have been shown, it will be apparent to those skilled in the art that the incisions may be non-radial, curved, or other shapes. When creating incisions 1518, it is important to keep the spatula or laser in substantially a single plane so as not to intersect and puncture the descemet or Bowman's membrane.

Once intrastromal incisions 1518 have been created, a fiber optic cable tip coupled to a fiber optic cable and a laser can be optionally inserted into each of the incisions 1518 for ablating tunnels 1526 to the desired size, if needed or desired. The laser beam emitted from the tip may be directed upon either first internal surface 1522, second internal surface 1524, or both for ablating tunnels 1526 to sequentially and incrementally remove three-dimensional portions from these surfaces. The laser source for the cable is advantageously similar to the laser source for the cable as discussed above. Alternatively, a drill or other suitable micro-cutting instruments can be used to sequentially and incrementally remove portions of the cornea.

Referring to FIG. 70, a plurality of radial tunnels 1526 are shown with a suitable tool 1550 projecting into one of the tunnels 1526 for introducing optical material 1528 into tunnels 1526 to modify cornea 1512. Ocular material 1528 as used herein refers to transparent fluids or solids or any combination thereof. In the examples of FIGS. 71-77, ocular material 1528 is a gel or fluid type material, which can be injected into pockets 1526 via tool 1550. Preferably, in this case, tool 1550 is a needle for injecting ocular material 1528 into pockets 1526. Of course as in the preceding embodiment, a solid implant or ocular material may be introduced into pockets 1526. Also, ocular material 1528 can have either a refractive index, which is different or the same as the intrastromal tissue of cornea 1512 as needed and/or desired, whether the ocular material is a gel, a solid or any combination thereof.

As shown in FIG. 71, optical material 1528 injected into the ablated tunnels 1526 expands the outer surface of cornea 1512 outward to change or modify the curvature of the central portion of cornea 1512 from its original shape shown in broken lines to its new shape shown in full lines.

As seen in FIGS. 71-77, the various radial tunnels 1526 can be filled with ocular material 1528 to overfill pockets 1526 (FIG. 71), underfill pockets 1526 (FIG. 72) or completely fill pockets 1526 (FIG. 73). Thus, by introducing various amounts of optical material into pockets 1526, the curvature of cornea 1512 can be varied at different areas. Similarly, selected tunnels 1526 can be overfilled or completely filled at selected areas, while other selected tunnels can be partially filled, completely filled or unfilled to collapse or decrease the curvature of cornea 1512 at other selected areas as shown in FIGS. 74-77. The selective alteration of the curvature in different areas of the cornea are particularly desirable in correcting astigmatisms.

In the embodiment illustrated in FIGS. 71-77, the intrastromal areas of tunnels 1526 are preferably ablated by a laser or cut by a micro-cutting instrument for sequentially and incrementally removing three-dimensional portions of cornea 1512 to form tubular pockets from tunnels 1526. However, as in the previous embodiment of FIGS. 61 and 62, the incisions 1518 can be filled with ocular material without previously ablating or cutting the internal surfaces 1522 and 1524 of cornea 1512 to expand the cornea 1512 for increasing its curvature. Ablating the internal surfaces of the cornea is advantageous to remove opaque areas of the cornea which can then be filled with the ocular material.

As shown in FIGS. 72 and 74, the amount of ocular material 1528 introduced into the ablated areas of pockets 1526 can be less then the amount of ablated material to reduce the curvature of cornea 1512. Alternatively, the amount of ocular material 1528 introduced into the ablated areas of pockets 1526 can completely fill pockets 1526 to retain the original curvature of cornea 1512 as seen in FIGS. 73, 74 and 75.

### Embodiment of FIGS. 78-81

Referring now to FIGS. 78-81, an eye 1610 is shown for treatment of hyperopia, myopia and/or removal of opaque portions in accordance with another embodiment of the invention using an implant or ocular material 1630. As shown, the eye 1610 includes a cornea 1612, a pupil 1614 and a lens 1616. As in the previous embodiments, the live eye 1610 is treated without freezing cornea 1612 or any part thereof.

In this embodiment, a thin layer 1618 of cornea 1612 is first removed from the center portion of a patient's live cornea 1612 by cutting using a scalpel or laser. The thin layer 1618 is typically on the order of about 0.2 mm in thickness with overall cornea being on the order of about 0.5 mm in thickness. Once the thin layer 1618 is removed from cornea 1612, it exposes first and second opposed internal surfaces 1622 and 1624. Generally, either or both of the internal surfaces 1622 and/or 1624 are the target of the ablation by the excimer laser. Alternatively, tissue from the internal surfaces 1622 and/or 1624 can be removed by a mechanical cutting mechanism, or substantially no tissue is removed from the cornea.

As illustrated in FIG. 78, a disc-shaped portion 1626 is removed from internal surface 1624 by a laser beam or other cutting mechanism. In this embodiment, internal surface 1624 is shaped to include a concave annular portion 1627. The method and laser apparatus as described above in the embodiment of FIGS. 25-34 can be used for removing tissue from cornea 1612 in substantially the same manner.

After the exposed internal surface 1622 or 1624 of cornea 1612 is ablated, if necessary, an annular ring shaped implant or ocular material 1630 is placed on ablated portion 1628 of cornea 1612. The previously removed thin layer 1618 of cornea 1612 is then replaced onto ablated portion 1626 of cornea 1612 to overlie implant or ocular material 1630 and then reconnected thereto. The resulting cornea can have a modified curvature thereby modifying the refractive power of the cornea and lens system as seen in FIGS. 79 and 80, or the original curvature with opaque areas removed and/or modified refractive power as seen in FIG. 81.

The ocular implant or material 1630 in the embodiment shown in FIGS. 78-81 has a substantially annular ring shape, and is substantially identical to the implant or ocular material 1430 discussed above. Thus, implant 1430 will not be illustrated or discussed in detail when referring to the procedures or methods of FIGS. 78-81.

The outer diameter of ocular implant or material 1630 can be about 3-9 mm, while the inner opening 1632 is generally about 1-8 mm. The thickness of ocular implant 1630 is preferably about 20 to about 1000 microns. Ocular implant 1630 has a planar face 1644 forming a frustoconically shaped surface, which faces inwardly towards the center of eye 1610 in a posterior direction of eye 1610 to contact the exposed inner surface 1620 of the cornea 1612. The opposite face 1646 is preferably a curved surface facing in an anterior direction of eye 1610 as shown. The ocular implant 1630 can be shaped to form a corrective lens or shaped to modify the curvature of the cornea. Similarly, the implant can be used to replace opaque areas of the cornea which have been previously removed by ablation or other means.

In the embodiment shown, ocular implant 1630 preferably has a substantially uniform shape and cross-section. Alternatively, ocular implant 1630 can be any suitable shape having either a uniform and/or non-uniform cross-section in selected areas as necessary to correct the patient's vision. For example, an ocular implant can be used having a circular or triangular cross section. In this manner, the curvature of a cornea can be modified at selected areas to correct various optical deficiencies, such as, for example, astigmatisms. Ocular implant 1630 can be a corrective lens with the appropriate refractive index to correct the vision of the patient. The ocular implant 1630 is made from a bio-compatible transparent material. Preferably, ocular implant 1630 is made from any suitable transparent polymeric material. Suitable materials include, for example, collagen, silicone, polymethylmethacrylate, acrylic polymers, copolymers of methyl methacrylate with siloxanylalkyl methylacrylates, cellulose acetate butyrate and the like. Such materials are commercially available from contact lens manufacturers. For example, optical grade silicones are available from Allergan, Alcon, Staar, Chiron and Iolab. Optical grade acrylics are available from Allergan and Alcon. A hydrogel lens material consisting of a hydrogel optic and polymethylmethacrylate is available from Staar.

Hydrogel ocular implant lenses can be classified according to the chemical composition of the main ingredient in the polymer network regardless of the type or amount of minor components such as cross-linking agents and other by-products or impurities in the main monomer. Hydrogel lenses can be classified as (1) 2-hydroxyethyl methacrylate lenses; (2) 2-hydroxyethyl methacrylate-N-vinyl-2-pyrrolidinone lenses; (3) hydrophilic-hydrophobic moiety copolymer lenses (the hydrophilic components is usually N-vinyl-2-pyrrolidone or glyceryl methacrylate, the hydrophobic components is usually methyl methacrylate); and (4) miscellaneous hydrogel lenses, such as lenses with hard optical centers and soft hydrophilic peripheral skirts, and two-layer lenses.

Alternatively, ocular implant 1630 can be elongated or arcuate shaped, disc shaped or other shapes for modifying the shape and curvature of cornea 1612 or for improving the vision of eye 1610 without modifying the curvature of cornea 1612. Similarly, ocular implant 1630 can be placed in the intrastromal area of the cornea 1612 at a selected area to modify the curvature of the cornea and correct the vision provided by the cornea and lens system. In the embodiment shown in FIGS. 78-81, thin layer 1618 of cornea 1612 is completely removed to expose the internal surfaces 1622 and 1624 of cornea 1612.

### Embodiment of FIG. 82

An alternative method of implanting ocular material or implant 1630 into an eye 1710 is illustrated in FIG. 82. Specifically, ocular material or implant 1630 is implanted into cornea 1712 of eye 1710 to modify the patient's vision. In particular, this method can be utilized for the treatment of hyperopia, myopia or removal of opaque portions of the cornea. As in the previous embodiments, the treatment of eye 1510 is accomplished without freezing cornea 1512 or any portion thereof.

In this method, a ring or annular incision 1718 is formed in cornea 1712 utilizing a scalpel, laser or any cutting mechanism known in the art. The scalpel, laser or cutting mechanism can then be used to cut or ablate an annular-shaped intrastromal pocket 1726 in cornea 1712 as needed and/or desired. Accordingly, an annular groove is now formed for receiving ocular material or implant 1630 which is discussed above in detail.

The annular groove formed by annular incision 1718 separates cornea 1712 into first and second opposed internal surfaces 1722 and 1724. First internal surface 1722 faces in the posterior direction of eye 1710, while second internal surface 1724 faces in the anterior direction of eye 1710. optionally, either internal surfaces 1722 or 1724 can be ablated to make the annular groove or pocket 1726 larger to accommodate ocular implant 1630.

The portion of cornea 1712 with internal surface 1722 forms an annular flap 1725, which is then lifted and folded away from the remainder of cornea 1712 so that ocular implant of material 1630 can be placed into annular pocket 1726 of cornea 1712 as seen in FIG. 82. Now, corneal flap 1725 can be folded over ocular implant or material 1630 and reconnected to the remainder of cornea 1712 via sutures or the like. Accordingly, ocular implant or material 1630 is now encapsulated within cornea 1712.

As in the previous embodiments, ocular implant or material 1630 can modify the curvature of the exterior surface of cornea 1712 so as to either increase or decrease its curvature, or maintain the curvature of the exterior surface of cornea 1712 at its original curvature. In other words, ocular implant or material 1630 can modify the patient's vision by changing the curvature of the cornea 1712 and/or removing opaque portions of the cornea and/or by acting as a corrective lens within the cornea.

### Embodiment of FIG. 83

Another embodiment of the present invention is illustrated utilizing ocular implant 1630 in accordance with the present invention. More specifically, the method of FIG. 83 is substantially identical to the methods discussed above in reference to FIGS. 78-81, and thus, will not be illustrated or discussed in detail herein. Rather, the only significant difference between the methods discussed regarding FIGS. 78-81 and the method of FIG. 83 is that the thin layer 1816 of FIG. 83 is not completely removed from cornea 1812 of eye 1810.

In other words, thin layer 1818 of cornea 1812 is formed by using a scalpel or laser such that a portion of layer 1818 remains attached to the cornea 1812 to form a corneal flap. The exposed inner surface 1820 of layer 1818 or the exposed internal surface 1824 of the cornea can be ablated or cut with a laser or cutting mechanism as in the previous embodiments to modify the curvature of the cornea. Ocular implant 1630 can then be placed between internal surfaces 1820 and 1824 of cornea 1812. The flap or layer 1818 is then placed back onto the cornea 1812 and allowed to heal. Accordingly, ocular implant 1630 can increase, decrease or maintain the curvature of eye 1810 as needed and/or desired as well as remove opaque portions of the eye.

### Embodiment of FIGS. 84 and 85

Referring now to FIGS. 84 and 85, an ocular implant or material 1930 in accordance with the present invention is illustrated for treatment of hyperopia or myopia. In particular, ocular implant or material 1930 is a disk shape member, which is as thin as paper or thinner. Ocular implant or material 1930 includes a center opening 1932 for allowing intrastromal fluids to pass between either sides of ocular implant or material 1930. Basically, ocular implant or material 1930 is constructed of a suitable transparent polymeric material utilizing diffractive technology, such as a Fresnel lens, which can be utilized to correct the focus of the light passing through the cornea by changing the refractive power of the cornea. Since ocular implant or material 1930 is very thin, i.e., as thin as paper or thinner, the exterior surface of the cornea will substantially retain its original shape even after ocular implant or material 1930 is inserted into the cornea. Even if there is some change in the cornea, this change can be compensated by the refractive powers of the ocular implant or material 1930.

Ocular implant or material 1930 can be inserted into the cornea in any of the various ways disclosed in the preceding embodiments. In particular, ocular implant or material 1930 can be inserted through a relatively small opening formed in the cornea by folding the ocular implant or material 1930 and then inserting it through the small opening and then allowing it to expand into a pocket formed within the intrastromal area of the cornea. Moreover, a thin layer or flap could be created for installing ocular implant or material 1930 as discussed above.

The outer diameter of ocular implant or material 1930 is preferably in the range of about 3.0 mm to about 9.0 mm, while center opening 1932 is preferably about 1 mm to about 8.0 mm depending upon the type of vision to be corrected. In particular, ocular implant 1930 can be utilized to correct hyperopia and/or myopia when using a relatively small central opening 1932 such as in the range of to about 1.0 mm to about 2.0 mm. However, if the opening is greater than about 2.0 mm, then the ocular implant or material 1930 is most likely designed to correct imperfections in the eye such as to correct stigmatisms. In the event of astigmatism, only certain areas of the ocular implant 1930 will have a refractive index which is different from the intrastromal tissue of the cornea, while the remainder of ocular implant or material 1930 has the same refractive index as the intrastromal tissue of the cornea.

Preferably, ocular implant 1930 is made from a biocompatible transparent material which is resilient such that it can be folded and inserted through a small opening in the cornea and then allowed to expand back to its original shape when received within a pocket in the cornea. Examples of suitable materials include, for example, substantially the same set of materials discussed above when referring to ocular implant or material 1430 or 1630 discussed above.

While various advantageous embodiments have been chosen to illustrate the invention, it will be understood by those skilled in the art that various changes and modifications can be made therein without departing from the scope of the invention as defined in the appended claims.

What is claimed is:

1. A method of treating a patient having presbyopia, comprising:
forming an incision in an outer surface of a patient's cornea;
creating an access path from the incision to an area within the cornea intersected by the main optical axis of the patient's eye;
forming a pocket within the cornea surrounding the main optical axis;
providing an annular ocular device having a peripheral portion with an outer diameter of between about 3.0 mm and about 9.0 mm and a central opening having a transverse size of about 2.0 mm or smaller, the ocular device being formed of a hydrogel;
positioning said ocular device in the pocket such that the main optical axis passes through said central opening; and
collapsing the pocket such that said live cornea encapsulates the annular ocular device and the shape of the annular ocular device influences the shape of the cornea to provide a refractive correction for the patient's eye.

2. The method of claim 1, wherein the annular ocular device comprises a pin hole aperture that enables light passing therethrough to be focused on the retina.

3. The method of claim 1, wherein at least one of (a) forming the incision, (b) creating the access path, and (c) forming the pocket comprises directing a laser at the cornea.

4. The method of claim 1, wherein the thickness of the ocular device is between about 20 microns and about 1000 microns.

5. The method of claim 1, wherein the peripheral portion of the ocular device comprises a posterior surface and an anterior surface, the anterior surface having a curvature configured to reshape the cornea to induce a refractive correction in the patient's eye.

6. The method of claim 5, wherein the posterior surface comprises a frustoconically shaped surface that faces inwardly toward the main optical axis of the eye.

7. The method of claim 5, wherein thickness as measured from the anterior surface to the posterior surface varies radially across the peripheral portion.

8. The method of claim 5, further comprising inserting a delivery tool through the incision and manipulating the delivery tool to urge the annular ocular device toward the pocket.

9. A method of compensating for presbyopia, comprising:
separating a layer of a patient's live cornea from the front of said live cornea;
moving said separated layer to expose an internal surface of said live cornea underneath said separated layer, a portion of said exposed internal surface being intersected by the main optical axis of the eye;
providing an ocular device having a peripheral portion configured to compensate for refractive error by modifying the curvature of the cornea and a central portion configured to compensate for decreased accommodation;
positioning said ocular device on said internal surface of said live cornea such that the main optical axis extends through said central portion; and
repositioning said separated layer of said live cornea back over said internal surface of said live cornea and said ocular device, such that the shape of said ocular device influences the shape of said repositioned separated layer of said live cornea.

10. The method of claim 9, wherein separating the layer of the cornea from the front of the cornea comprises directing a laser toward the cornea.

11. The method of claim 9, wherein separating the layer of the cornea from the front of the cornea comprises forming a pocket in the cornea.

12. The method of claim 9, wherein separating the layer of the cornea from the front of the cornea comprises forming a flap of corneal tissue.

13. The method of claim 9, further comprising removing corneal tissue adjacent to the exposed internal surface prior to positioning the ocular device.

14. The method of claim 9, wherein the ocular device comprises a flexible ring-shaped member.

15. The method of claim 14, wherein the ring-shaped member has a central hole configured to permit intrastromal fluids to pass therethrough.

16. The method of claim 14, wherein the ring-shaped member has a central pin hole such that light from objects over a substantial range of distances from the eye is focused on the retina.

17. The method of claim 9, wherein the ocular device comprises a material having a refractive index that substantially matches that of a layer of the cornea.

18. The method of claim 9, wherein the ocular device comprises a material having a refractive index different from that of an intrastromal layer of the cornea.

19. The method of claim 9, wherein positioning comprises injecting the ocular device onto the internal surface.

20. The method of claim 9, wherein the ocular device comprises a hydrogel.

21. A method of treating a patient having refractive error, comprising:
forming an incision in an outer surface of a patient's cornea;
creating an access path from the incision to an area within the cornea intersected by the main optical axis of the patient's eye;
providing an annular ocular device having a peripheral portion with an outer diameter of between about 3.0 mm and about 9.0 mm and a central opening having a transverse size of about 2.0 mm or smaller;
positioning said ocular device in the pocket such that the main optical axis passes through said central opening; and
collapsing the pocket such that said live cornea encapsulates the annular ocular device and the shape of the annular ocular device influences the shape of the cornea to provide a refractive correction for the patient's eye.
